(19) United States
(12) Patent Application Publication
(10) Pub. No.: US 2014/0030712 A1
(43) Pub. Date: Jan. 30, 2014

(54) GENOMIC APPROACH TO THE IDENTIFICATION OF BIOMARKERS FOR ANTIBIOTIC RESISTANCE AND SUSCEPTIBILITY IN CLINICAL ISOLATES OF BACTERIAL PATHOGENS

(71) Applicants: E. Lynn Zechiedrich, Houston, TX (US); Michelle C. Swick, Houston, TX (US); Richard S. Sucgang, Houston, TX (US)

(72) Inventors: E. Lynn Zechiedrich, Houston, TX (US); Michelle C. Swick, Houston, TX (US); Richard S. Sucgang, Houston, TX (US)

(73) Assignee: BAYLOR COLLEGE OF MEDICINE, Houston, TX (US)

(21) Appl. No.: 13/983,195

(22) PCT Filed: Feb. 1, 2012
(86) PCT No.: PCT/US2012/023490

§ 371 Date: Oct. 21, 2013

Related U.S. Application Data
(60) Provisional application No. 61/438,459, filed on Feb. 1, 2011.
(60) Provisional application No. 61/469,085, filed on Mar. 29, 2011.
(60) Provisional application No. 61/543,874, filed on Oct. 6, 2011.

(51) Int. Cl. C12Q 1/68

(52) U.S. Cl. CPC C12Q 1/689
USPC 435/6.11

(57) ABSTRACT

In certain embodiments, the present invention concerns genotypic identification of bacteria that are resistant to a bacteria and subsequent determination of an appropriate therapy. In specific embodiments, a high-throughput genotypic detection method for biomarkers for antibiotic resistance and susceptibility allows efficient prescription practice and increases the likelihood of a successful therapeutic outcome. In certain embodiments, the information from the genotypic detection method is utilized for determining antibiotics that should be avoided or, alternatively, employed.

This application claims priority to U.S. Provisional Patent Application Ser. No. 61/438,459, filed Feb. 1, 2011, and to U.S. Provisional Patent Application Ser. No. 61/469,085, filed Mar. 29, 2011, and to U.S. Provisional Patent Application Ser. No. 61/543,874, filed Oct. 6, 2011, all of which applications are incorporated by reference herein in their entirety.

### STATEMENT REGARDING FEDERALLY SPONSORED RESEARCH OR DEVELOPMENT

This invention was made with government support under RO1A1054830 and T32 GM88129 awarded by the National Institutes of Health. The government has certain rights in the invention.

### TECHNICAL FIELD

The field of the invention includes at least microbiology, cell biology, molecular biology, and medicine. In specific aspects the field of the invention includes antibiotic resistance and methods and compositions related thereto.

### BACKGROUND OF THE INVENTION

Multidrug resistance in bacterial pathogens is an increasing public health threat that is compounded by a lack of new antibacterial agents. Although gram-positive organisms such as methicillin-resistant Staphylococcus aureus (MRSA) capture headlines, gram-negative pathogens are emerging with resistance to nearly every existing antibiotic. Patients presenting with symptoms of bacterial infection are treated empirically, before the presence of bacteria is verified or the antibiotic susceptibility of the pathogen is determined. As a consequence, antibiotics are prescribed that may not be necessary or effective against the infection. Because both pathogens and normal flora are exposed to these antibiotics, the long-term result of this practice is widespread multidrug resistance. Although several factors influence prescription decisions (current hospital formulary, personal preference, drug cost, marketing trends), the choice is made without the relevant knowledge of the pathogen.

More rapid detection of an MDR pathogen could make the difference between successful treatment and death. Most drug susceptibility and bacterial detection methods are based on phenotype and take several days to reveal a drug resistance profile. A genotypic method would reduce the time for diagnosis and improve the appropriateness of therapy. Rapid species identification options are available, but they do not report drug susceptibility, and are still expensive to implement into the diagnostic environment. Some plasmid detection assays are in use, but these assays detect only specific drug resistance mechanisms and are still minimally available in clinical diagnostic laboratories.

It is clear, however, that much of what causes drug-resistance remains largely unknown and the previous candidate drug target gene approach has not resulted in better diagnostics. If, however, the pathogen could be characterized completely through a high-throughput method that screens for markers of drug resistance mechanisms (such as SNPs or sequences), both plasmid and chromosomal, the physician would know precisely what antibiotics hold the highest promise for successful treatment in a timely fashion. Similar antibiotic resistance phenotypes should be reflected in genotype. Toward that end, the inventors used a strategy to pool clinical isolates with similar drug resistance phenotypes to reveal genomic fingerprints of resistance.

### BRIEF SUMMARY OF THE INVENTION

In embodiments of the invention, there are genomic fingerprints that correspond to antibiotic resistance phenotypes in clinical isolates, for example, including methods of identifying resistant bacteria and developing a treatment therapy based on genotypic information about the bacteria (in certain embodiments, as opposed to phenotypic information). In particular embodiments, the present invention concerns molecular mechanisms, clinical trends and genomic fingerprints in multidrug-resistant isolates, including, for example, E. coli isolates. In some embodiments, the present invention allows diagnostics of bacterial multi-drug resistance (MDR) based on genotype to the effect of at least rendering faster, easier, and more accurate diagnosis, eliminate empirical prescribing of antibiotics, and/or preserving antibiotic efficacy.

In some embodiments of the invention, there is a new next generation sequencing (NGS) approach based on clustering bacterial clinical isolates by antibiotic-resistance phenotypes and sequencing the resultant pooled genomic DNA.

In embodiments of the invention, the methods concern a genotypic assay based on DNA sequence variations linked to antibiotic resistance. In embodiments of the invention, there are methods that utilize a combined pooling, sequencing, and SNP-subtraction based approach to identify SNPs associated with susceptibility or resistance of bacterial pathogens for a particular antibiotic. One can group clinical isolates into pools based on similarity in patterns of susceptibility or resistance to one or more antibiotics (for example, by k-means clustering). Sequencing data can be generated (for example only, with next generation sequencing (NGS) techniques) for each pool and compared to selected bacterial reference genome(s). Variations at the whole genome level of the drug-susceptible pools align to the genome of exemplary drug-susceptible laboratory strains, whereas those of multidrug-resistant pools are more similar to multidrug resistant environmental isolates, in certain embodiments. In embodiments of the invention, genomic footprints of antibiotic susceptibility as well as antibiotic resistance are identified. Relative to certain reference strains, SNPs encoding nonsynonymous changes in protein sequences in common among all pools of antibiotic resistant isolates may be located in particular genes, such as those involved in DNA metabolism, in specific embodiments (such as gyrA, libB, mutM, and/or recG). In the representative examples for gyrA, libB, mutM, and/or recG, these genes are tightly linked in the exemplary E. coli pan-genome and a gyrA variant occurs only when accompanied by two or three of these variants, indicating that they are involved in development of antibiotic resistance, in certain embodiments. The present invention provides methods for identifying genomic fingerprints related to antibiotic resistance diagnostics.

Certain embodiments of the invention allowed identification of particular features of the E. coli (as a representative bacteria) pan-genome: (i) conserved SNPs correlate with antibiotic resistance phenotypes of the pools; (ii) regions of high levels of genome variation among clinical isolate E. coli correspond to large genomic rearrangements (inversions, amplifications) that occurred between the two most diverged E. coli genomes known; (iii) SNPs are biallelic 99.2% of the time and triallelic the remaining 0.8% of the time, and in no instance do all four possible nucleotides occur at any given nucleotide; (iv) extremely tightly linked and novel SNPs conserved across the E. coli pangenome accompany the well-known the exemplary gyrase mutations that lead to fluoroquinolone resistance (as a representative example of a bacterial phenotype).

In some embodiments, the invention provides a framework for new diagnostic based upon antibiotic resistance genotype. In specific aspects, high divergence of one pool pushes species boundary. In some particular cases, 3% of (6 Gb) sequence matches nothing in GenBank®, which allows new avenues for exploration. In at least certain cases, prophage sequences previously proposed to be important for fluoroquinolone resistance are absent from many clinical isolates. In some embodiments, genomic fingerprints are useful not only for drug resistance, but also drug susceptibility, for example using a SMS-3-5 reference genome. In particular aspects, a fluoroquinolone resistance genomic fingerprint encompasses genes involved in DNA repair.

In embodiments of the invention, a SNP subtraction platform that the inventors developed can be used to analyze any large dataset of genomic sequences to uncover SNPs associated with specific phenotypes. In addition to antibiotic resistance or susceptibility, other exemplary phenotypes include temperature, UV radiation, heavy metal resistance, pH, sugar and nucleotide metabolism, salt tolerance, osmolarity, replication ability/rates, biofilm, species boundary identification, media biases, conjugation, tranduction efficiencies, secretion systems, riboswitches, microbiome identification, antibacterial vaccines, growth speed, pigment production, accelerated gene expression, cell size, cooperativity, metabolite production, infectivity, and/or radioresistance (in specific embodiments, any phenotype that can be quantified is amenable to this type of pooling).

Bacteria related to the methods and compositions of the invention may be of any kind, including gram positive and gram negative. The bacteria may be resistant to one or more drugs. In specific aspects of the invention, the pathogen is subjected to a high-throughput method that screens for markers of drug resistance mechanisms (such as SNPs or sequences), both plasmid and chromosomal, allowing the health care provider to determine what antibiotic(s) may be employed for successful treatment in a timely fashion. SNPs may be distributed over genic and non-genic regions of the chromosome, and the SNPs may be located in regions not previously associated with resistance.

Resistance of the bacteria to an antibiotic may have occurred by any methods, including at least drug inactivation or modification (for example, enzymatic deactivation by β-lactamases); alteration of a target site (for example, alteration of a binding target site of the drug); alteration of a metabolic pathway (for example, some sulfonamide-resistant bacteria do not require para-aminobenzoic acid (PABA), an important precursor for the synthesis of folic acid and nucleic acids in bacteria inhibited by sulfonamides; and reduced drug accumulation (for example, by decreasing drug permeability and/or increasing active efflux from the cell surface).

Embodiments of the invention also include methods of identifying genotypes in bacteria that may be then used to determine suitable antibiotic therapy, analogous to exemplary methods described herein for E. coli.

Examples of antibiotics to which the bacteria may become resistant to include aminoglycosides, ansamycins, carbacephem, carbapenems, cephalosporins, glycopeptides, lincosamides, lipopeptide, macrolides, monobactams, nitrofurans, penicillins, polypeptides, quinolones, fluoroquinolones (including Ciprofloxacin, Gatifloxacin, Levofloxacin, Norfloxacin), sulfonamides, tetracyclines, sulfa drugs, and/or drugs against mycobacteria.

The antibiotics to which the bacteria become resistant may be bactericidal antibiotics or bacteriostatic antibiotics, for example.

Although the present disclosure illustrates the present invention with specific embodiments to E. coli, the present invention is also useful in a similar context to other bacteria, including, but not limited to, the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also be useful against gram negative bacteria such as Klebsiella pneumoniae, Escherichia coli, Proteus, Serratia species, Acinetobacter, Yersinia pestis, Francisella tularensis, Enterobacter species, Citrobacter, Bacteriodes and Legionella species and the like. In addition, the invention may prove useful in controlling protozoan or macroscopic infections by organisms such as Cryptosporidium, Isospora belli, Toxoplasma gondii, Trichomonas vaginalis, Cyclospora species, for example, and for Chlamydia trachomatis and other Chlamydia infections such as Chlamydia psittaci, or Chlamydia pneumoniae, for example.

In some embodiments, there is a method of determining a genotype of a bacteria, said genotype associated with resistance to one or more antibiotics, comprising the steps of: comparing genomic sequence of a bacteria susceptible to at least one particular drug with genomic sequence of a bacteria resistant to at least the drug; and identifying at least one genetic marker that correlates with resistance of the drug. Any method of the invention may include obtaining a sample from an individual, whether or not that sample is obtained directly from the individual or upon storage or transportation following removal from the individual. In a specific embodiment, the genetic marker comprises a single nucleotide polymorphism (SNP). In some embodiments, the information from the method is employed in the determination of therapy for an individual known to have a bacterial infection or suspected of having a bacterial infection.

In some embodiments, there is a method of determining selection of an antibiotic drug for an individual in need thereof, comprising the steps of: providing an individual with one or more symptoms of a bacterial infection; obtaining a sample from the individual, said sample comprising bacteria that causes the infection; identifying a genotype from the bacteria, wherein said genotype provides information about resistance or susceptibility to one or more antibiotic drugs; and employing the information in the selection of treatment of the individual. In a specific embodiment, the individual has been diagnosed with a bacterial infection. In specific embodiments, the infection is a deleterious infection to the health of the individual. In specific embodiments, the individual is infected with or suspected of being infected with Escherichia coli, including pathogenic E. coli.

In some embodiments, there is a method of determining resistance or susceptibility of one or more bacteria to one or more antibiotics, comprising the steps of: obtaining or providing a plurality of bacteria of the same species; sequencing a nucleic acid region from the plurality of bacteria; comparing the sequence to the corresponding sequence of a reference bacteria of the same species, said reference bacteria known to be resistant or susceptible, respectively, to the one or more antibiotics; and identifying differences, similarities, or both between the bacteria from the plurality with the reference bacteria.

In some embodiments, there is a method of determining resistance or susceptibility of one or more bacteria to one or more antibiotics, comprising the steps of: grouping a plurality of bacteria based on known patterns of susceptibility or resistance to one or more antibiotics; sequencing nucleic acid from each of the bacteria in the plurality; comparing the sequence of the nucleic acid to a corresponding nucleic acid sequence from a reference bacteria of the same species, said reference bacteria known to be resistant or susceptible, respectively, to the one or more antibiotics; identifying a genomic fingerprint for the plurality that represents a respective genotype for the susceptibility or resistance. In specific embodiments, the genomic fingerprint comprises one or more SNPs that are common among at least the majority of the plurality. In certain aspects, the SNPs are located in DNA metabolism genes. In specific embodiments, the antibiotic is selected from the group consisting of aminoglycosides, ansamycins, carbacephem, carbapenems, cephalosporins, glycopeptides, lincosamides, lipopeptide, macrolides, monobactams, nitrofurans, penicillins, polypeptides, quinolones, fluoroquinolones, sulfonamides, tetracyclines, sulfa drugs, drugs against mycobacteria, and a combination thereof.

In some embodiments of the invention, information from the method (including for example determination of resistance or susceptibility of a bacteria) is employed in diagnosis of a pathogenic bacteria from an individual. In specific embodiments, the method further comprises obtaining a sample from the individual, such as mucus, sputum, saliva, feces, blood, nasal swab, throat swab, or a mixture thereof. In certain embodiments, the sequencing is further defined as next generation sequencing.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1. Data analysis workflow. Sequence reads were assembled into contigs and mapped to reference genomes DH10B (susceptible) and SMS-3-5 (resistant) to generate consensus genome fingerprints for each antibiotic resistance profile. SNP analysis continued from the fingerprints to detect SNP markers corresponding to resistance phenotype. Contigs which mapped to neither reference genome to passed to de novo assembly and identified using BLAST searches as either known sequences belongs to plasmids, phage or other bacterial species, or as novel sequences. One can have visual display of results showing the source of sequence as either E. coli, non-E. coli, or novel sequence.

FIG. 2 shows analysis of exemplary E. coli reference genomes currently available in National Center for Biotechnology Information (NCBI) by phylogenetic analysis.

FIG. 3 shows for the exemplary reference DH10B a plot of HQ SNPs for each pool along the position on the chromosome.

FIG. 4. A) Resistance specific SNPs broken down by pool. B) Genes affected by resistance specific, genic SNPs. Genic SNPs were mapped to the 4,357 known genes in the DH10B reference genome. SNPs that were identical in identity and position were denoted high-quality (HQ) SNPs, whereas SNPs that matched in position only were denoted as high-confidence (HC) SNPS. C) HQ SNPs do not correlate with pool size. The total number of HQ SNPs detected in each pool was plotted vs. pool size. A best-fit line to these data showed a very weak negative correlation (R2=0.0907).

FIG. 5. A) HQ SNPs relative to reference genomes. The total number of HQ SNPs relative to each reference genome DH10B and SMS-3-5 reported for each genomic fingerprint. B) Similarity of genomic fingerprint to each reference genome. The logarithm of SNPs_{DH10B}/SNPs_{SMS-3-5} was plotted relative to the number of drug classes to which each consensus resistance phenotype was resistant. non-MDR denotes classes to which isolates were resistant to fewer than 3 drug classes. MDR indicates resistance to 3 or greater drug classes. C) Phylogenetic analysis of each genomic fingerprint in context of each reference genome.

FIG. 6. SNP frequency plots aligned with mummer plot of DH10B and SMS-3-5.

FIG. 7 shows cluster strains by antibiotic resistance phenotype.

FIG. 8 shows cluster strains by antibiotic resistance phenotype in which certain strains were removed in the analysis.

FIG. 9 illustrates an exemplary sequence analysis strategy.

FIG. 10 illustrates SNPs relative to the exemplary drug-susceptible strain DH10B.

FIG. 11 shows exemplary SNPs associated with antibiotic resistance.

FIG. 12 shows identifying SNPs for both resistance and susceptibility.

FIGS. 13 and 14 show examples of generating a genomic fingerprint for antibiotic resistance.

FIG. 15 demonstrates an exemplary genomic fingerprint for fluoroquinolone resistance.

FIG. 16. Exempalry data analysis workflow. Sequence reads were mapped to reference genomes DH1 OB (susceptible), REL606 (susceptible) and SMS-3-5 (multidrug resistant) to generate pool consensus sequence genome fingerprints for each antibiotic resistance profile. SNP analysis continued from the fingerprints to detect SNP markers corresponding to resistance phenotype. Sequence reads that mapped to neither reference genome were passed to de novo assembly, followed by analysis.

FIG. 17. SNP frequency plots to reference genome DH10B. The frequency of SNPs in the consensus sequence of each pool along the chromosome was plotted against the DH1 OB reference genome. The y-axis for each pool was altered to visualize where the SNPs occurred. Peaks in the plot represent regions of high variability for a pool. “H” pools are the top three plots; “S” pools are the bottom two plots.

FIG. 18. Representation of prophage in E. coli clinical isolates. (a) Prophage coverage in pools. The assembled consensus sequence of each pool was probed for the presence of cryptic prophage. Coverage (y-axis) was normalized to the average coverage of DH1 OB core genes and displayed by intensity for each prophage in each pool (black, low coverage; red higher coverage). (b) Presence of prophages in fluoroquinolone-susceptible and fluoroquinolone-resistant clinical isolates. Individual isolates were tested for the presence of prophage genes intR for rae, and perR for CP4-6 by PCR. The percentage of isolates testing positive for each gene is shown for all fluoroquinolones-susceptible (n=18) and fluoroquinolone-resistant (n=65) isolates tested. These isolates were chosen from pools from the full range of coverage shown in (A).

FIG. 19. SNPs associated with fluoroquinolone susceptibility and resistance. Unanimous SNPs that result in nonsynonymous changes in genes were computed relative to each of three reference genomes, one multidrug resistant (SMS-3-5), and the other two susceptible (DH1 OB and REL606). Variant genes shared among reference genomes are represented in Venn diagrams. (a) Genes carrying SNPs that correlate with a susceptible phenotype (SNPs that occurred in any fluoroquinolone-resistant pool sequence were subtracted from those that occurred in all fluoroquinolone-susceptible pool sequences). Underlined genes are encoded on an SMS-3-5-specific plasmid (pSMS35_{—}130). (b) Genes carrying SNPs that correlate with a fluoroquinolone-resistant phenotype (SNPs that occurred in any fluoroquinolone-susceptible pool sequence were subtracted from those that occurred in all fluoroquinolone-resistant pool sequences).

FIG. 20. Linkage analysis of the mutM, JigS, and recG fluoroquinolone resistance associated SNPs. (a) Relative chromosomal location of mutM, radC, ligB, spoT, and recG in E. coli DH1 OB. (b) To determine the frequency with which any two SNPs (or three SNPs in one case) are both present or both absent in E. coli, 39 complete E. coli genomes in GenBank® and 83 draft genomes from the Broad Institute were probed for the presence or absence of SNPs uncovered by the subtraction analysis; mutM, ligB, and recG. SNPs in radC and spoT were detected by variation analysis of the same set of genomes and selected as controls based on their location between the genes of interest. The frequency of co-occurence of each SNP pair was plotted along a range from never linked (−1) to always linked (+1). Strains with unannotated genes were not included in the analysis.

### DETAILED DESCRIPTION OF THE INVENTION

Antibiotic-resistant bacterial pathogens are a grave threat to public health. The increasing prevalence of gram-negative bacteria resistant to nearly every existing antibiotic is of particular concern because of a dearth of new antimicrobial agents¹.

Gram-negative infections comprise the bulk of nosocomial infections in the US. Each year, ˜2 million people develop bacterial infections while in the hospital², and more than half of these infections involve bacteria that are multidrug-resistant^{1,2}. The cost to treat multidrug-resistant infections is ˜30% more than drug-susceptible infections, totaling 21 to 34 billion USD annually. Antibiotic resistance is promoted by exposure of pathogens as well as the normal microbiota to antibiotics³. Patients presenting with symptoms of bacterial infection are often treated empirically, before the presence of bacteria is verified or antibiotic susceptibility determined, which takes several days⁴. As a consequence, antibiotics are prescribed that may not be necessary or effective. Bacterial identification based upon genotype would be faster, improve the accuracy of diagnosis, increase the likelihood for successful treatment, and curb unnecessary antibiotic exposure. Genotypic species identification is becoming more affordable and accessible, but genotypic determination of antibiotic susceptibility is not yet in use in the clinic. Assays to detect plasmid-borne antibiotic resistance genes exist⁵, but are not widely used in clinical settings.

Antibiotic resistance is complex, with both mobile genetic elements and chromosomal genes contributing to resistance, and can be conferred by many different mechanisms⁶. While comparative genomics on individually sequenced strains reveal variations in known resistance genes, natural variation between the strains can create so much background that the discovery of novel resistance mechanisms by this method is difficult. A pool of isolates that share a resistance phenotype should also share genomic signatures. Sequencing pools of isolates provides insight into the evolution of antibiotic resistance and may uncover new antibiotic resistance mechanisms.

Detection and identification of bacteria and determination of antibiotic susceptibility currently relies on culturing the pathogen and takes a few days. Meanwhile, physicians are forced to treat based upon empirical observations, a practice that not only negatively impacts the likelihood of successful treatment outcome, but also promotes antibiotic resistance. With few new antibiotics in the pipeline, and none for gram negative pathogens, one must preserve the existing antibiotic arsenal. DNA sequencing technology no longer requires culturing and would thus allow rapid identification of variations at a genomic scale. The inventors set out to determine genomic changes associated with antibiotic resistance toward the goal of a more rapid and accurate diagnostic platform. Instead of sequencing individual genomes, they elected to sequence pools of isolates with similar antibiotic resistance phenotypes. In this way, the inventors dampened genetic variations in individual isolates and highlighted variations that the pooled isolates had in common. They identified variants linked to fluoroquinolone resistance that were overlooked previously by traditional approaches. Moreover, they uncovered additional genomic variations that may promote antibiotic susceptibility. The data provide the foundation for a rapid, accurate way to diagnose antibiotic resistance, and the methods are useful to be applied to any large genomic datasets linked to disease, for example.

Using next generation sequencing technologies, in certain embodiments, the inventors have generated genomic fingerprints correlated with antibiotic resistance phenotypes in clinically isolated E. coli. Such fingerprints serve to combat the growing epidemic of multidrug resistant bacterial infections caused in part by empirical use of antibiotics. Genomic DNA sequences were mapped to the exemplary drug-susceptible DH10B and the multidrug-resistant SMS-3-5. Coverage averaged 150× and SNPs were identified with high confidence. SNPs correlated strongly with antibiotic resistance; the majority fall in regions of the chromosome not previously associated with antibiotic resistance. The antibiotic-resistant pools exhibited significantly fewer polymorphisms relative to SMS-3-5, indicating an environmental reservoir for MDR mechanisms. The identified SNPs with strong linkage to antibiotic resistance phenotypes represent a powerful collection of potential biomarkers that can be used to guide antibiotic therapy.

Exemplary Enterobacter genera that are encompassed in the invention include at least the following: Alishewanella; Alterococcus; Aquamonas; Aranicola;Arsenophonus; Azotivirga; Rlochmannia; Brenneria; Buchnera; Budvicia; Buttiauxella; Cedecea; Citrobacter; Cronobacter; Dickeya; Edwardsiella; Enterobacter; Erwinia, e.g. Erwinia amylovora, Erwinia tracheiphila, Erwinia carotovora etc.; Escherichia, e.g. Escherichia coli; Ewingella; Grimoniella; Hafnia; Klebsiella, e.g. Klebsiella pneumonia; Kluyvera; Leclercia; Leminorella; Moellerella; Morganella; Obesumbacterium; Pantoea; Pectobacterium see Erwinia; Candidatus Phlomobacter; Photorhabdus, e.g. Photorhabdus luminescens; Poodoomaamaana; Plesiomonas, e.g. Plesiomonas shigelloides; Pragia; Proteus, e.g. Proteus vulgaris; Providencia; Rahnella; Raoultella; Salmonella; Samsonia; Serratia, e.g. Serratia marcescens; Shigella; Sodalis; Tatumella; Trabulsiella; Wigglesworthia; Xenorhabdus; Yersinia, e.g. Yersinia pestis; and Yokenella.

Current diagnostics of bacterial MDR are based on phenotype. Following extraction of a sample from an individual, bacteria are cultured, and after species identification and antibiotic resistance determination and the individual may have already received one or more doses of an empirically-prescribed antibiotic. A method based on genotype would be beneficial, allowing elimination of guesswork with empirical prescribing, reducing the delay for species identification and generation of antibiotic resistance profile, reducing exposure of pathogens (and normal biota) to antibiotics, and increasing the likelihood of successful treatment outcome.

### EXAMPLES

The following examples are included to demonstrate preferred embodiments of the invention. It should be appreciated by those of skill in the art that the techniques disclosed in the examples which follow present techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. The scope of the appended claims is not to be limited to the specific embodiments described.

### Example 1

### Overview of Exemplary Strategy

Since 1999, the inventors have collected more than 6,000 E. coli clinical isolates from patients treated for infection at Ben Taub General Hospital in the Texas Medical Center, located in Houston, Tex. These isolates represent fluoroquinolone MICs spanning seven orders of magnitude and a wide range of phenotypes as derived from hospital antibiogram data of drug susceptibility status to 22 antibiotics (Table 1). 164 non-clonal isolates from unique patients, representing all the resistance phenotypes existing in the collection, were stratified into 16 pools by k-means clustering, ranging in pool size from 2-33 isolates and in phenotype from susceptible to all tested antibiotics to nearly pan-drug resistant (Table 3). Genomes were represented within each pool at equimolar concentrations.

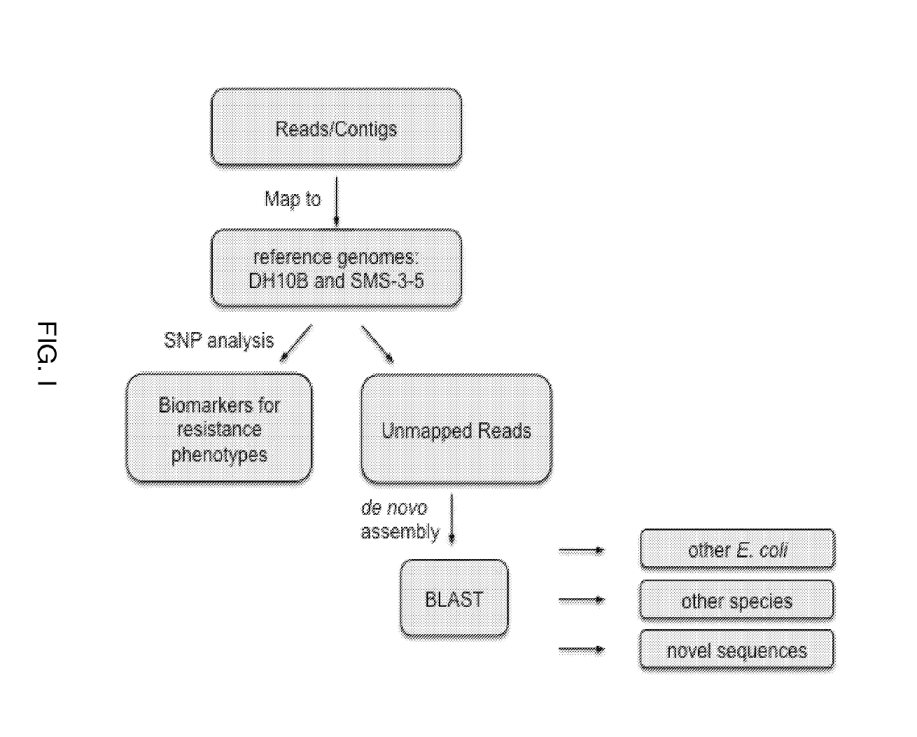

Pooled DNA was sequenced using the SOLiD 3 platform. Sequence reads were assembled into contigs. Contigs ranged in size from 3441 to 24859 bp and averaged between 241 and 491 bp, depending on the pool. The average contig size and N50 value for each pool are reported in Table 2. Following the workflow diagrammed in FIG. 1, contigs were mapped onto reference genomes. With coverage per base of 20-400× and averaging 150×; high quality (HQ) single nucleotide polymorphisms (SNPs) were identified and de novo assembly was performed with very high confidence (p<1.0×10⁻¹⁹). SNPs were considered HQ only when they were called by the analysis tools as identical in position and nucleotide identity for all isolates within a pool. The assumption of diploidy inherent in the analysis tools may have removed SNPs that occurred in less than one out of seven genomes in a pool. Thus, HQ SNPs were enriched in the pool.

### Example 2

### SNP Analysis

Mapped to the reference DH10B, a well-characterized, 4.6 Mb genome laboratory strain susceptible to all antibiotics, the inventors detected a total of 252,333 SNPs; approximately 9.4% were HQ. The inventors plotted these HQ SNPs for each pool along the position on the chromosome, starting with the origin of replication (FIG. 3). Several pools show low occurrence of SNPs across the chromosome while others showed specific regions of high SNP frequency. Pool H01, which had fluoroquinolone MICs of >1,000 μg/ml (Table 3), showed high frequency SNPs across the length of the genome and alone, accounted for 33% of the total number of SNPs detected. This extremely high divergence suggests that H01 is highly divergent from typical E. coli, despite passing biochemical tests. SNP analysis was not possible with this high divergence, so H01 was omitted from the rest of this section. Below the inventors describe the analysis of its genome sequence.

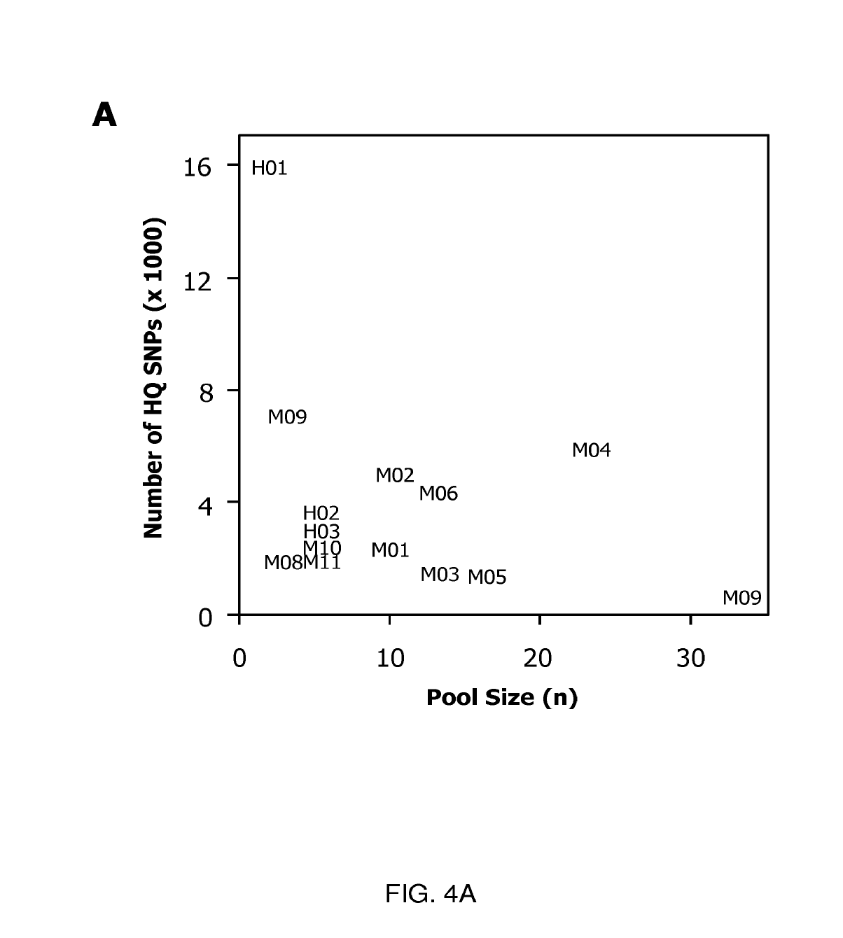

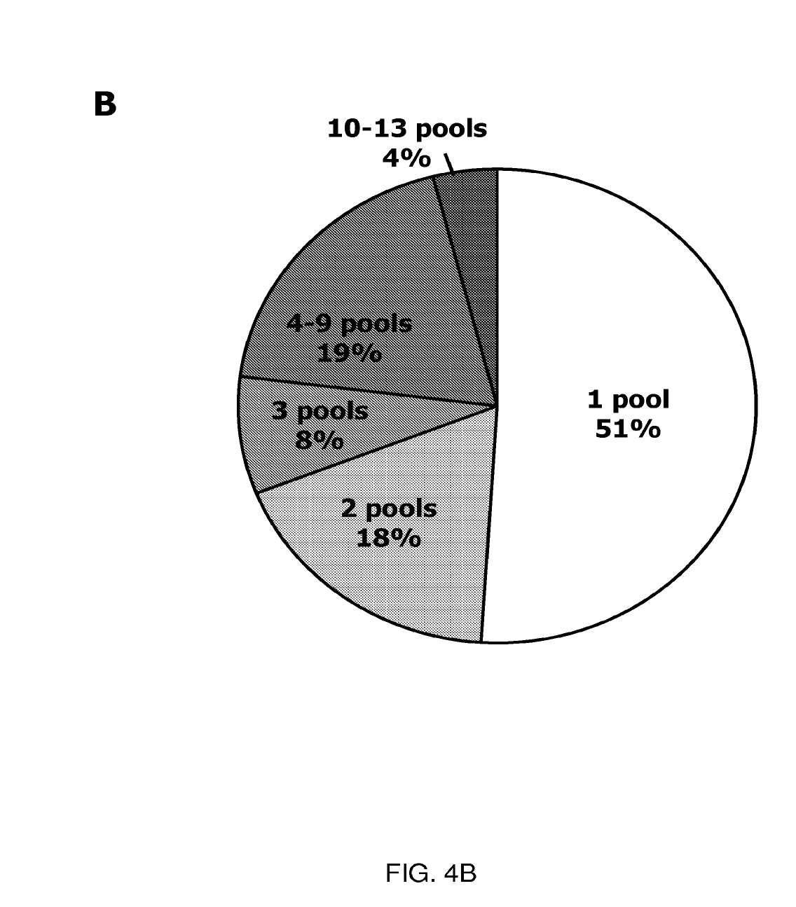

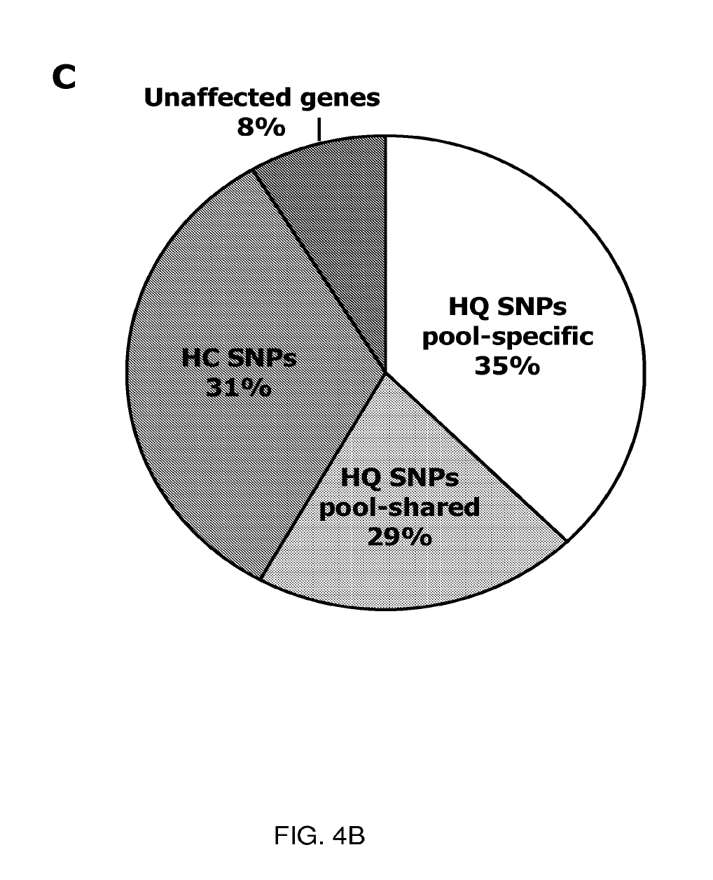

Given the extremely high sequencing coverage, a single SNP even in a pool with 33 different isolates (Table 3) should be detected. In order to meet the criterion of conservation across a pool, one might expect a general decrease in HQ SNPs with increasing number of genomes in the pool. FIG. 4A shows, however, that total number of SNPs and pool size did not correlate. Pool H01 was determined to be an outlier by the Extreme Studentized Deviate test statistic. The best-fit line for the remaining pools had a slope of −0.0424 and a correlation coefficient of 0.194.

Of all the HQ SNPs, 80% were found within genic regions, which represent ˜85% of the chromosome. The remaining SNPs were found in non-genic regions, in which we include regions of unknown coding status. In agreement with previous results, homotypic conversions (purine to purine or pyrimidine to pyrimidine) occurred twice as often as heterotypic SNP conversions. Even allowing for this 2-fold preference for homotypic conversions, HQ SNPs were overwhelmingly diallelic in the dataset. 99.2% of all HQ SNPs were only one of two possible nucleotides, 0.8% were one of three, and never did all four nucleotides occur at any SNP. This diallelism allowed the inventors to filter SNPs common to both drug-susceptible (S) from drug-resistant pools (M and H) to enrich for SNPs specific to antibiotic resistance.

˜4% of the remaining SNPs were in common to all remaining pools, showing conserved differences between the clinical isolates and DH10B. About 25% of SNPs were shared among 2, 3, or 4 pools, and about half were unique to and invariant in a single pool (FIG. 4B). Of the 4,357 annotated genes in the DH10B genome, only 8% were identical between the reference genome and clinical isolates, (FIG. 4C), 31% of genes from the reference by high confidence (HC) SNPs (those that occurred in a pool, but were not absolutely conserved throughout the pool). HQ pool-shared SNPs occurred in about a quarter of the genes, and 35% of the protein-encoding genes contained HQ SNPs that were pool-specific (FIG. 4C). In certain embodiments, 80% SNPs fall in genic regions; 1-3 pool SNPs represent ˜75% of the data; genic SNPs affected 92% of genes; and/or shared pool SNPs mark commonalities in pool phenotypes. In specific embodiments, SNPs represent genomic fingerprints of antibiotic resistance and, in certain embodiments, the vast majority in the sequences are not known to be associated with antibiotic resistance.

A number of chromosomal mutations have been linked with antibiotic resistance. As examples, mutations in the gyrA gene of gyrase (S83L and D87Y/N) and the parC gene of topoisomerase IV (S80I and E84K/G) occur ubiquitously in fluoroquinolone-resistant bacteria. The S80I mutation in parC in pool M03 as a HQ SNP. The inventors detected mutations resulting in S83L of gyrA and S80I of parC in the other fluoroquinolone-resistant pools.

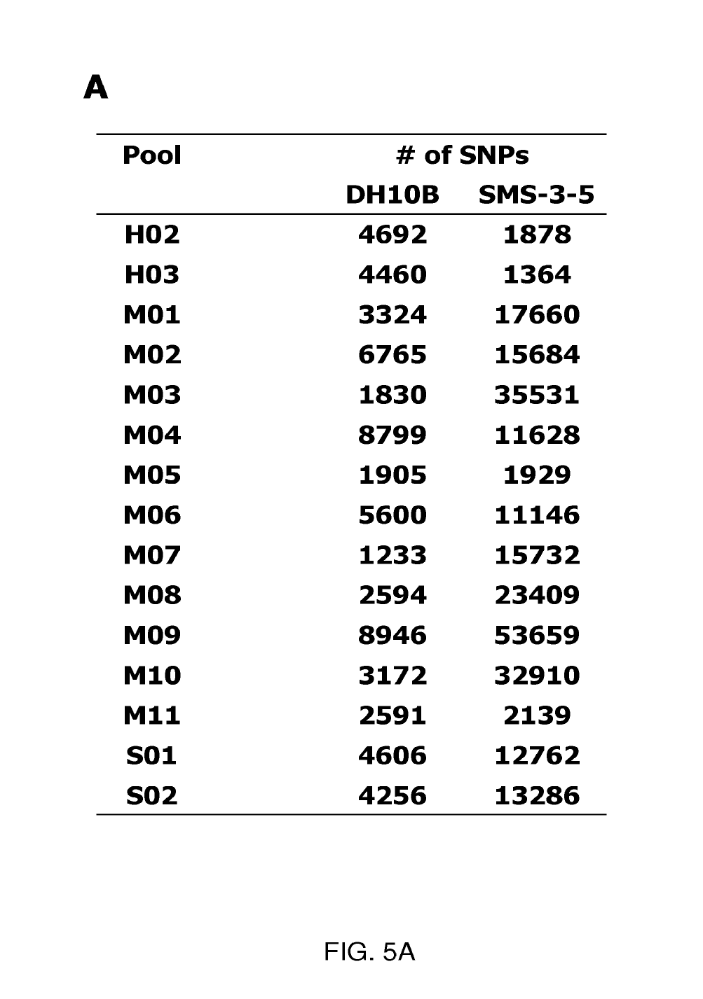

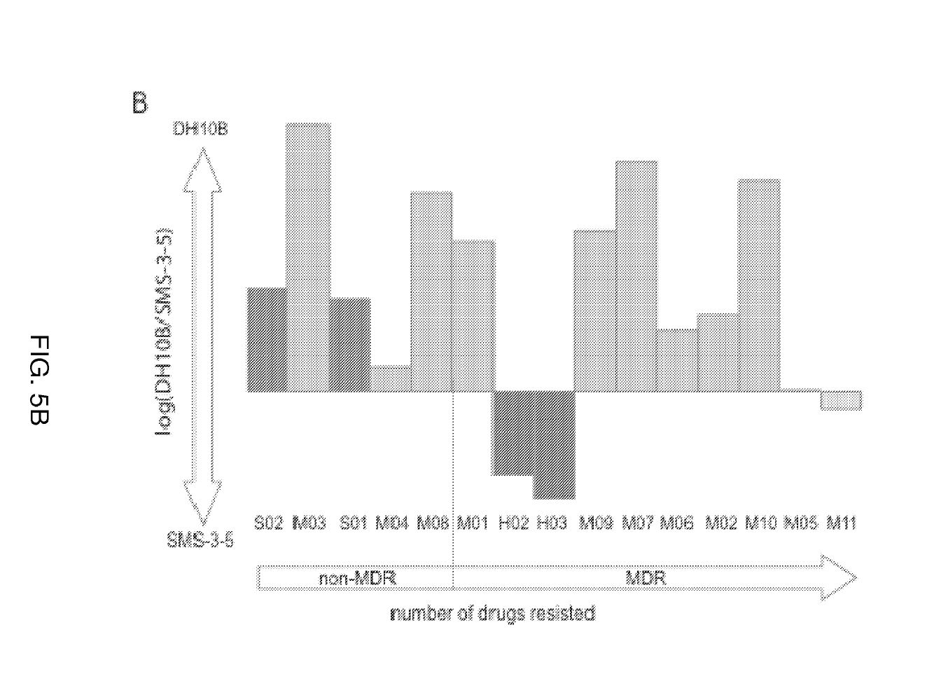

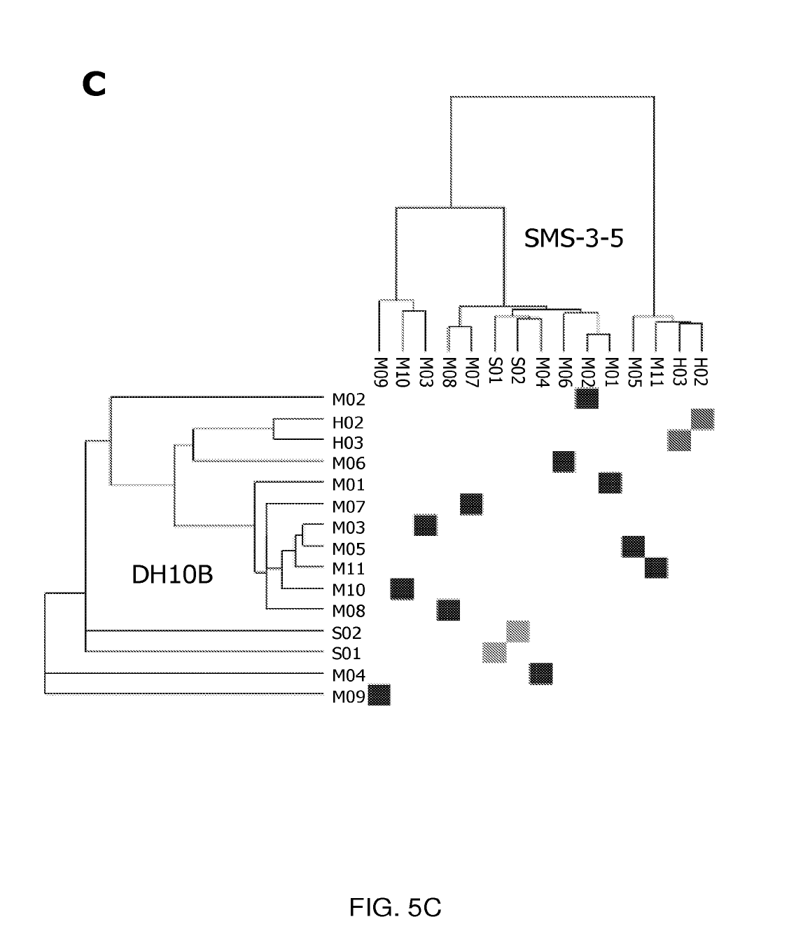

FIG. 5A demonstrates HQ SNPs relative to reference genomes; the total number of HQ SNPs relative to each exemplary reference genome DH10B and SMS-3-5 were reported for each genomic fingerprint. There was similarity of genomic fingerprint to each reference genome (FIG. 5B). The logarithm of SNPs_{DH10B}/SNPs_{SMS-3-5} was plotted relative to the number of drug classes to which each consensus resistance phenotype was resistant, and non-MDR denotes classes to which isolates were resistant to fewer than 3 drug classes. MDR indicates resistance to 3 or greater drug classes. In FIG. 5C, there is phylogenetic analysis of each genomic fingerprint in context of each reference genome.

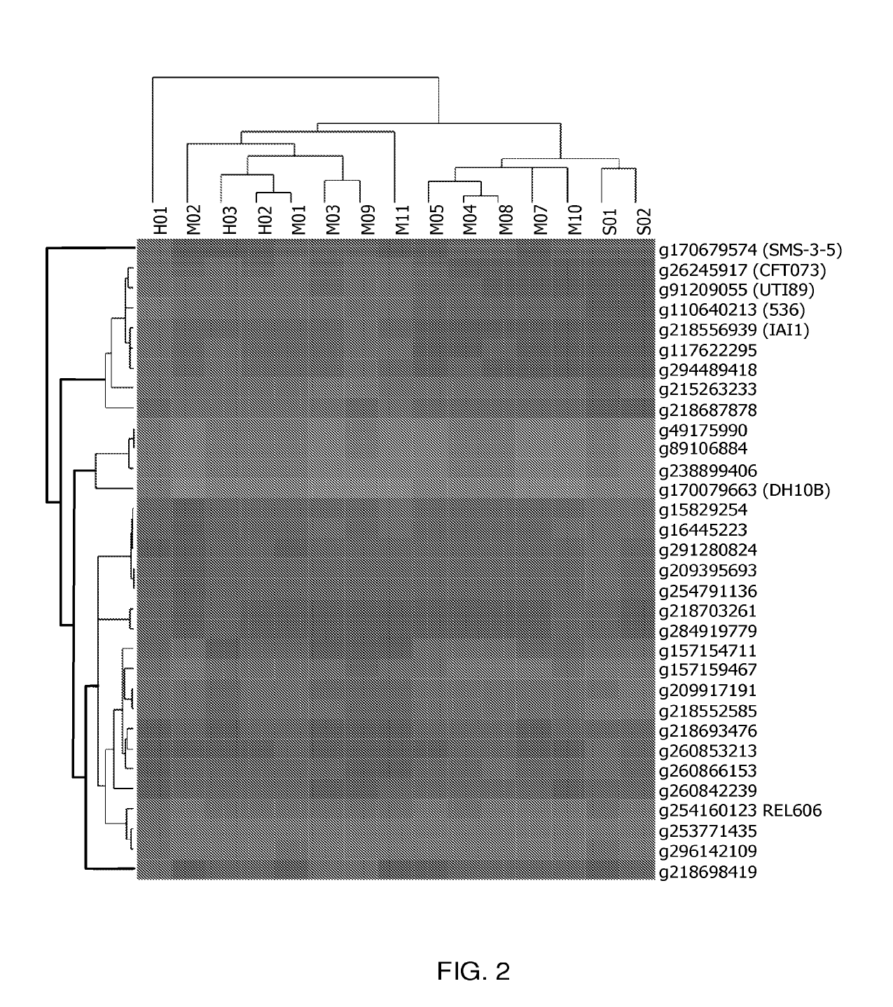

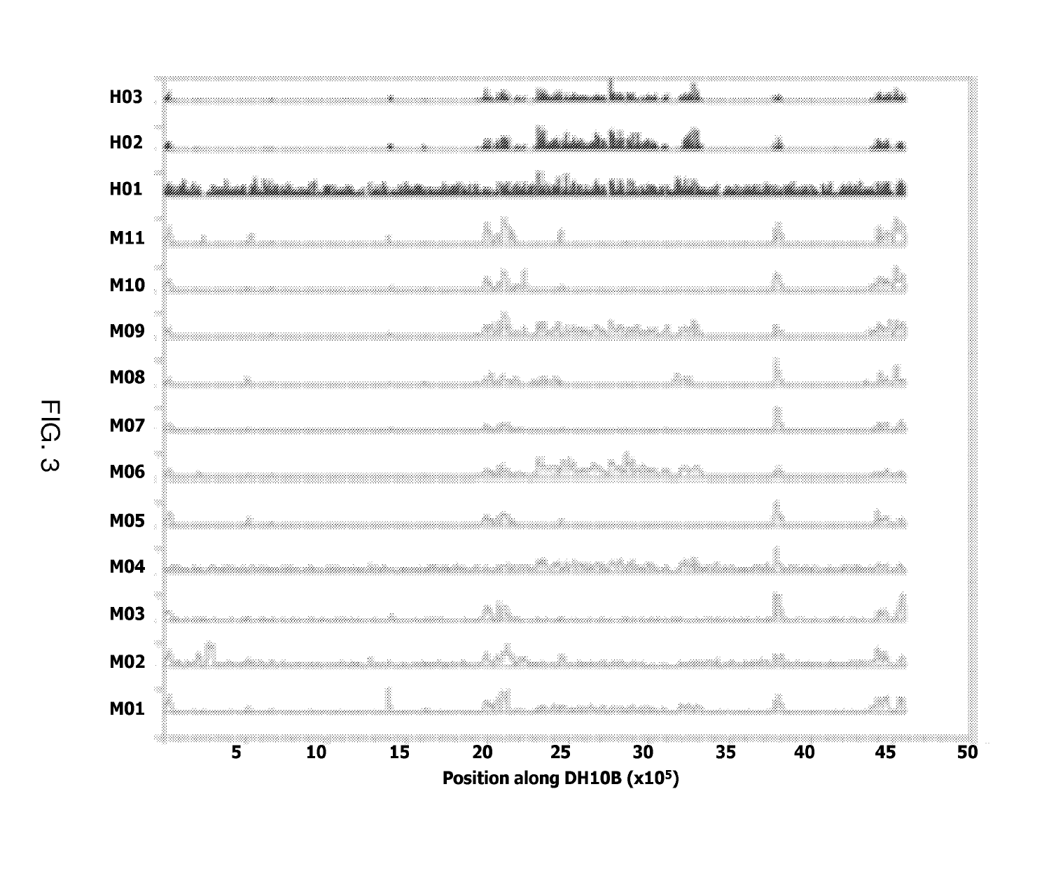

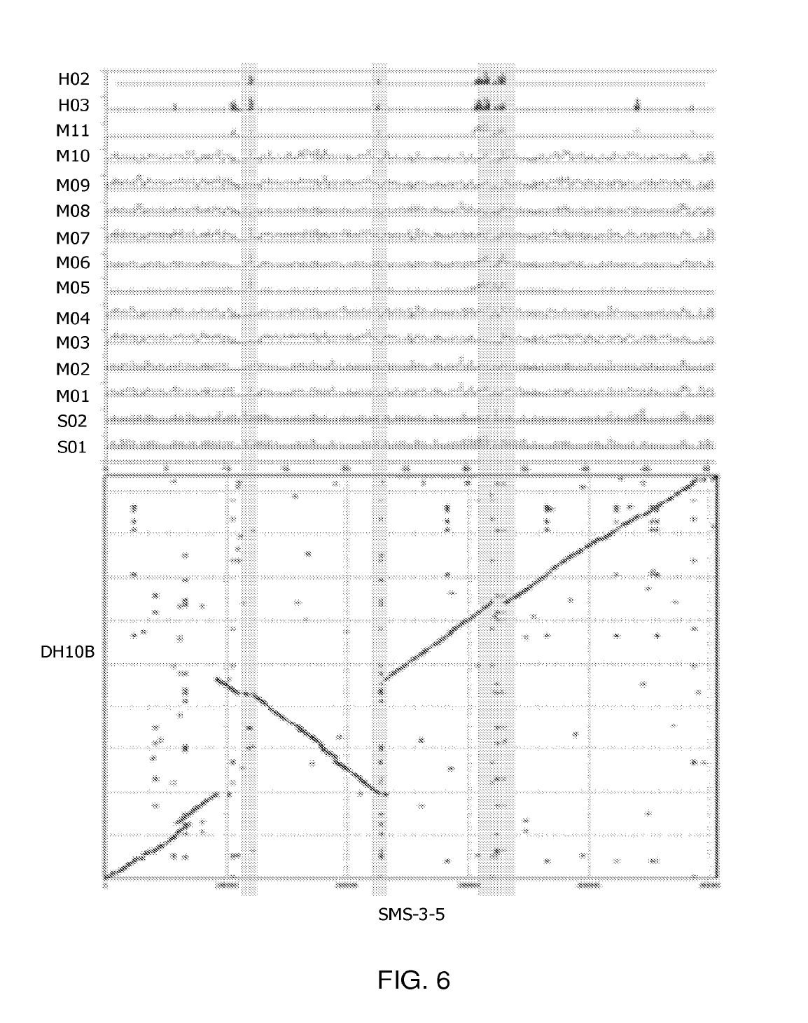

In an attempt to map the remaining one-third of the unmapped contigs, the inventors analyzed the 33 E. coli reference genomes currently available in NCBI by phylogenetic analysis (FIG. 2). The 5.1 Mb genome of the multidrug-resistant soil isolate SMS-3-5 was the most divergent sequence from the other E. coli genomes, and mapped well to additional sequences in all pools, especially those with high fluoroquinolone MICs. SMS-3-5 was reported to have extremely high fluoroquinolone MICs and was also resistant to 32 of 33 tested compounds from a wide range of drug classes. Pools M05, M11, and the H pools, exhibited far fewer SNPs to the SMS-3-5 genome than to the DH10B genome (FIG. 6). These pools and SMS-3-5 were comparably resistant to record concentrations of drugs, including the fluoroquinolones. In contrast to these drug-resistant pools, the drug-susceptible pools showed high SNP frequencies relative to the SMS-3-5 genome (FIG. 6). In specific embodiments, chromosomal regions with high occurrence of SNPs align with major genomic changes.

### Example 3

### De Novo Analysis

Contigs that did not map to the two reference genomes were used for de novo assembly. Using BLAST, these contigs were matched to currently known genes in the E. coli pangenome, as well as sequences from other bacterial species, plasmids, and phages.

Nine cryptic prophages were recently reported to play important physiological roles in growth, biofilm formation, stress response, and antibiotic resistance in a K12 strain. The inventors investigated the presence and prevalence of prophage sequences in the pools. Surprisingly, the prophage sequences were overall poorly covered relative to the surrounding regions of the genome/overall coverage of the full genome. Particularly, the prophage implicated in quinolone resistance, ras, was one of the least detected in most pools.

Novel sequences composed ˜3% of the unmapped data and may be new resistance genes or become part of the genomic fingerprint of their pool.

Pool H01 contained only 2 isolates, but was highly variable from the reference genomes. All the contigs were used for de novo assembly for this pool. Notably, the contigs of this pool were, on average, dramatically longer than contigs of any other pool, even pools containing similarly few genomes. The resulting genome sequence was 6.7 Mb in length, 1.1 Mb longer than the longest E. coli genome in GenBank®.

### Example 4

### Significance of Certain Embodiments of the Invention

The key mutations in Gyrase and Topoisomerase IV, despite having a high frequency of occurrence in fluoroquinolone-resistant isolates, were not detected using the high stringency data filter the inventors used for analysis, a filter that would be necessary and appropriate in the diagnostic setting. The “black-and-white” test is something that is necessary to move genotypic detection into the clinic, but our genomic analysis shows something much less simple. For example, in each the D87Y/N of Gyrase and the E84K/G in Topoisomerase IV, two possible SNPs may occur to result in the resistant mutant. Thus, a diagnostic test for one would miss isolates harboring the other SNP. In cases such as this, it seems that instead, screening for the absence of the wild type nucleotides might prove to be the better diagnostic. This idea brings up a new concept—one of biomarkers for susceptibility. In embodiments of the invention, there is a powerful detection method that involves both screening for SNP of antibiotic resistance and SNPs for antibiotic susceptibility.

A soil isolate that was likely never exposed to antibiotics, SMS-3-5, exhibited record high MICs, particularly for flouroquinolones, and these MICs were higher than any reported in the clinic. However, standardized microbiology laboratory protocols measure only the breakpoint MIC for each fluoroquinolone (4-16 ug/ml). The inventors reported fluoroquinolone MICs for several isolates that were also so high that a modified broth dilution scheme had to be implemented to measure them (Boyd et al. 2009). The genomes of these isolates were remarkably similar to the SMS-3-5 genome, supporting the hypothesis that soil bacteria are a reservoir for antibiotic resistance.

Embodiments of the invention provide an alternative to the current phenotypic methods used to determine antibiotic resistance. By reducing the wait of generation of an antibiotics resistance profile, a high-throughput genotypic detection method for biomarkers for antibiotic resistance and antibiotic susceptibility would eliminate the guesswork of empirical prescription practice and increase the likelihood of successful treatment outcome. The high incidence SNPs uncovered here, along with the strong linkage of SNPs to antibiotic resistance phenotypes, are a powerful collection of biomarkers that can be used to guide future antibiotic therapy.

### Example 5

### Exemplary Methods

Reagents and chemicals. Mueller-Hinton (MH) broth was from Difco (Sparks, Md.); tryptone and yeast extract were from Becton Dickinson and Company (San Jose, Calif.); gentamycin was from Sigma-Aldrich; PureLink™ Pro 96 Genomic DNA Kit was from Invitrogen (Carlsbad, Calif.). NanoDrop® Spectrophotometer ND-1000 was from Thermo Scientific (Wilmington, Del.). Oligonucleotide primers, Taqman probes, and Taqman Master Mix were from Applied Biosystems (Carlsbad, Calif.).

Clinical isolate collection and antibiotic resistance determination. E. coli clinical isolates collected from Ben Taub General Hospital over a seven-year span (1999-2006) that were not clonal, came from unique patients, and represented all drug resistance phenotypes with a set of >6,000 strains, were characterized previously using a candidate-gene approach (SKML, LBB, MCS). Hospital-derived qualitative antibiotic susceptibility status denoted each isolate as susceptible (S), intermediate resistant (I), or resistance (R) to the drugs listed in Table 1. Quantitative MICs for the fluoroquinolones ciprofloxacin (CIP), gatifloxacin (GAT), levofloxacin (LVX), and norfloxacin (NOR) were determined in our laboratory as described (LBB).

Sequencing pool design. Data from 214 representative clinical isolates were grouped into pools according to the combined qualitative and quantitative susceptibility data using k-means clustering. 32 total pools were generated. By manual inspection, we removed pools with only one strain, pools without sufficient data to justify to leave 16 pools (Table 3) that were chosen for whole-genome sequencing. The phenotypes ranged from susceptible to all drugs tested to nearly pan-drug resistant. Pools were denoted “S” when fluoroquinolone MICs were susceptible; M when they ranged in between a certain range, and “H” under certain other conditions but with norfloxacin MICs>1000 μg/ml.

Genomic DNA isolation and pool assembly. Genomic DNA was isolated from each isolate using the PureLink™ Pro 96 Genomic DNA Kit and quantified using a NanoDrop® Spectrophotometer ND-1000. All DNA samples had A₂₆₀/₂₈₀ greater than 1.8. Genomic DNA was pooled according to pool design such that each isolate was equally represented.

SOLiD™ Sequencing. The inventors sequenced the genomic DNA of each pool using 2×25 bp mate-paired libraries with the Applied Biosystems SOLiD™ System according to the manufacturers' instructions. Briefly, between 16-45 ug of DNA per library were sheared to 2.0 kb using the Covaris™ S2 System according to manufacturers' instructions. Genomic EcoP15I restriction enzyme sites were methylated prior to EcoP15I CAP Adaptor ligation. Samples were then size selected and circularized incorporating the internal adaptor. In the subsequent EcoP15I restriction enzyme step, the DNA was cleaved 25-27 bp away from the unmethylated enzyme recognition site in the CAP adaptor forming the DNA mate-pair. Finally, P1 and P2 adaptors were ligated to the mate-paired libraries for PCR amplification.

Each library template was clonally amplified on SOLiD P1 beads using emulsion PCR. Templated (P2 positive) beads were then enriched and deposited on an octet of a slide. SOLiD sequencing was carried out at 2×25 bp, using SOLiD v3.5 chemistry according to manufacturer's instructions.

TABLE 1
List of exemplary drugs tested to generate
drug resistance phenotypes.

| Antibiotic | Antimicrobial Class |
|---|---|
| Norfloxacin | Fluoroquinolones |
| Ciprofloxacin | |
| Gatifloxacin | |
| Levofloxacin | |
| Amikacin | Aminoglycosides |
| Gentamycin | |
| Tobramycin | |
| Ceftriaxone | Cephalosporins |
| Ceftazidime | |
| Cefotetan | |
| Cefepime | |
| Cefoxitin | |
| Cefotaxime | |
| Cefazolin | |
| Aztreonam | Monobactams |
| Imipenem | Carbapenems |
| Amoxicillin-clavulanic acid | Combination Pencillins |
| Piperacillin-tazobactam | |
| Ticarcillin-clavulanic acid | |
| Nitrofurantoin | Nitrofurans |
| Ampicillin | Penicillins |
| Sulfamethoxazole-trimethoprim | Combination Synthetics |

TABLE 2
Characteristics of pool contigs.

| Pool | n | Total Contigs | Avg. Length | Max. Length | N₅₀ |
|---|---|---|---|---|---|
| S01 | 9 | 8,509 | 291 | 98,016 | 7,809 |
| S02 | 9 | 6,763 | 262 | 50,755 | 2,037 |
| M01 | 10 | 7,605 | 293 | 60,772 | 7,553 |
| M02 | 10 | 7,591 | 308 | 49,999 | 5,992 |
| M03 | 13 | 3,441 | 397 | 49,421 | 8,127 |
| M04 | 23 | 8,693 | 240 | 33,514 | 1,885 |
| M05 | 16 | 7,856 | 296 | 54,690 | 5,912 |
| M06 | 13 | 5,594 | 361 | 107,844 | 10,511 |
| M07 | 33 | 6,661 | 301 | 50,652 | 4,743 |
| M08 | 3 | 7,581 | 283 | 71,047 | 5,454 |
| M09 | 3 | 24,859 | 250 | 59,232 | 630 |
| M10 | 5 | 5,713 | 365 | 68,273 | 8,111 |
| M11 | 5 | 5,534 | 374 | 91,897 | 8,948 |
| H01 | 2 | 4,512 | 491 | 198,167 | 31,155 |
| H02 | 5 | 6,618 | 282 | 48,966 | 4,720 |
| H03 | 5 | 6,148 | 374 | 98,194 | 11,969 |

TABLE 3
Antibiotic resistance phenotypes of sequenced pools.

| | | | Fluoroquinolone MIC, μg/ml | | | |
|---|---|---|---|---|---|---|
| Pool | n | Consensus Resistance Phenotypes | CIP | GAT | LVX | NOR |
| S01 | 9 | ampicillin, sulfamethoxazole- | 0.01 | 0.01-0.02 | 0.02-0.07 | 0.03-0.09 |
| no MDR | | trimethoprim | | | | |
| S02 | 9 | none | 0.01-0.09 | 0.01-0.09 | 0.03-0.17 | 0.04-0.34 |
| no MDR | | | | | | |
| M01 | 10 | fluoroquinolones, ampicillin, | 30-100 | 10-30 | 20-50 | 200-700 |
| MDR ≧3 | | cefoxitin | | | | |
| M02 | 10 | fluoroquinolones, ampicillin, | 100-500 | 10-30 | 20-50 | 200-700 |
| MDR ≧5 | | cefoxitin, cefazolin, gentamycin, | | | | |
| | | sulfamethoxazole-trimethoprim, | | | | |
| | | ticarcillin-clavulanic acid | | | | |
| M03 | 13 | fluoroquinolones | 10-30 | 8-30 | 8-30 | 50-200 |
| no MDR | | | | | | |
| M04 | 23 | fluoroquinolones, ampicillin | 100-400 | 30-100 | 50-100 | 200-700 |
| no MDR | | | | | | |
| M05 | 16 | fluoroquinolones, ampicillin, | 20-50 | 10-20 | 30-50 | 50-300 |
| MDR ≧5 | | cefoxitin, sulfamethoxazole- | | | | |
| | | trimethoprim, ticarcillin- | | | | |
| | | clavulanic acid | | | | |
| M06 | 13 | fluoroquinolones, ampicillin, | 50-200 | 10-20 | 30-100 | 200-400 |
| MDR ≧3 | | gentamycin, sulfamethoxazole- | | | | |
| | | trimethoprim | | | | |
| M07 | 33 | fluoroquinolones, ampicillin, | 10-30 | 10 | 10-20 | 50-200 |
| MDR ≧3 | | amoxicillin-clavulanic acid, | | | | |
| | | sulfamethoxazole-trimethoprim | | | | |
| M08 | 3 | fluoroquinolones, | 50-200 | 100-300 | 50-200 | 100-200 |
| No MDR | | sulfamethoxazole-trimethoprim | | | | |
| M09 | 3 | fluoroquinolones, ampicillin, | 20-30 | 20 | 10-20 | 100-200 |
| MDR ≧3 | | gentamycin | | | | |
| M10 | 5 | fluoroquinolones, ampicillin, | 100-400 | 20-100 | 30-100 | 50-500 |
| MDR ≧5 | | nitrofurantoin, sulfamethoxazole- | | | | |
| | | trimethoprim, ticarcillin- | | | | |
| | | clavulanic acid | | | | |
| M11 | 5 | fluoroquinolones, ampicillin, | 10-100 | 10-50 | 20-50 | 50-200 |
| MDR ≧3 | | nitrofurantoin, sulfamethoxazole- | | | | |
| | | trimethoprim | | | | |
| H01 | 2 | all tested except amikacin and | 100-300 | 10-30 | 10-200 | 1000 |
| MDR ≧5 | | imipenem | | | | |
| H02 | 5 | fluoroquinolones, ampicillin, | 50-500 | 10-30 | 40-50 | 400-1000 |
| MDR ≧3 | | cefoxitin | | | | |
| H03 | 5 | fluoroquinolones, amoxicillin- | 200-500 | 50-200 | 100 | 400-1000 |
| MDR ≧3 | | clavulanic acid, nitrofurantoin, | | | | |
| | | gentamycin | | | | |

CIP = ciprofloxacin
GAT = gatifloxacin
LVX = levofloxacin
NOR = norfloxacin

### Example 6

### Exemplary Workflow for Antibiotic Resistance Cluster Analysis

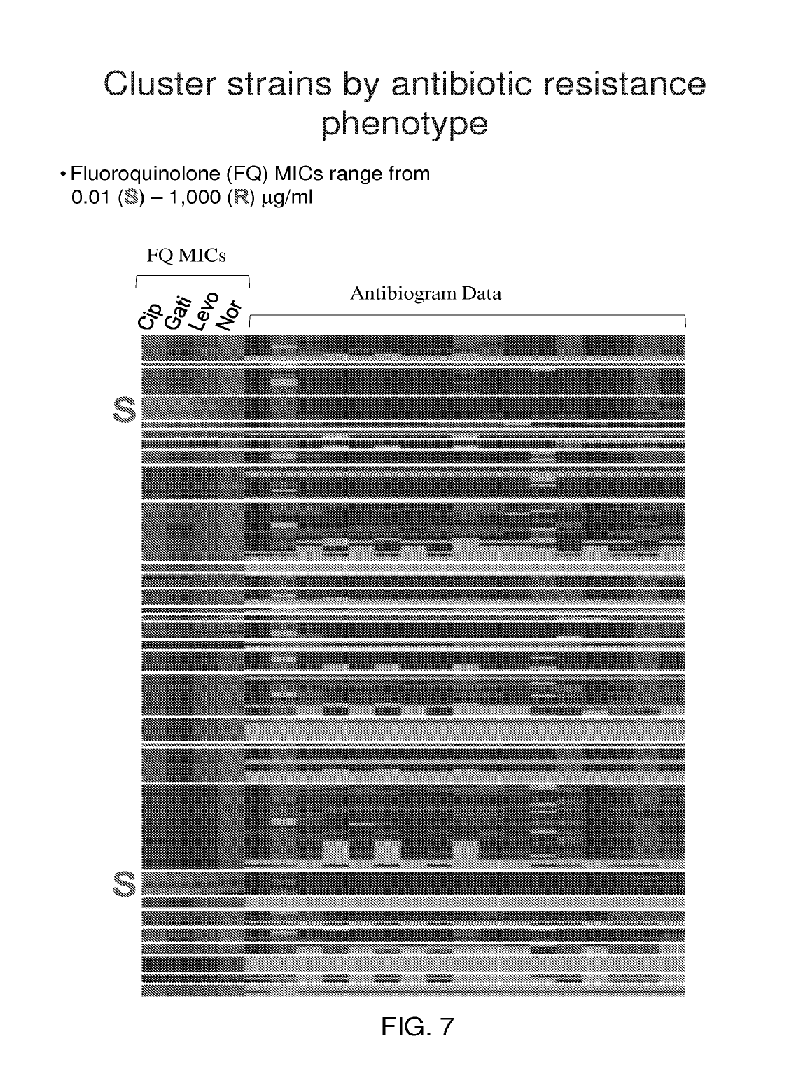

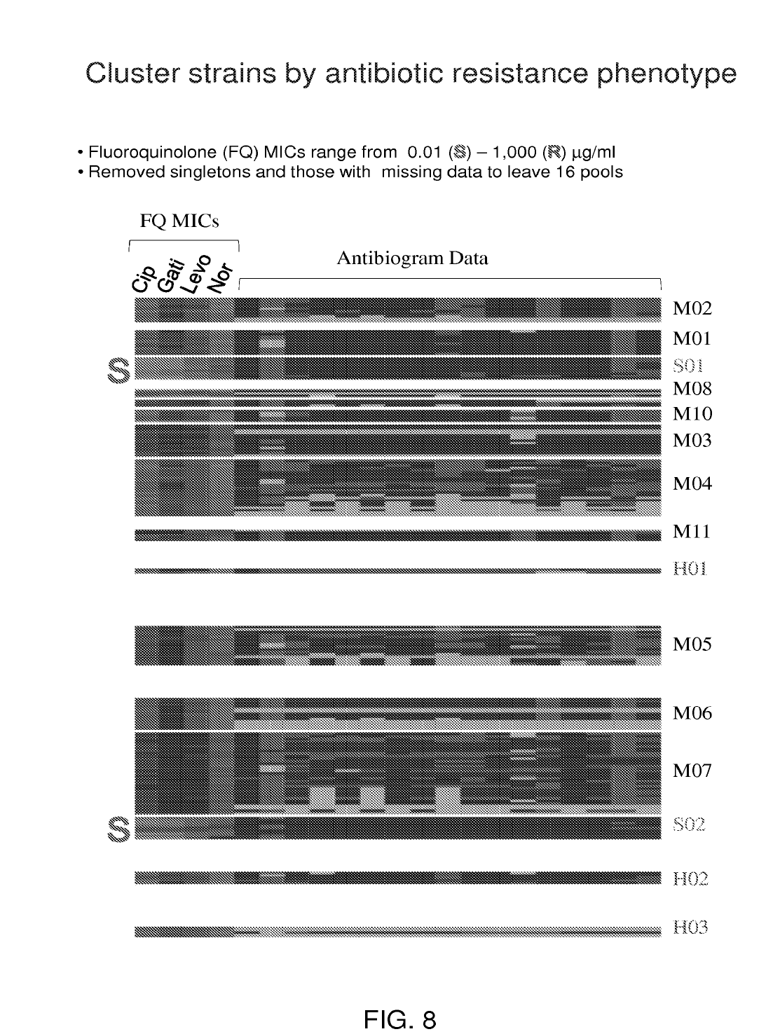

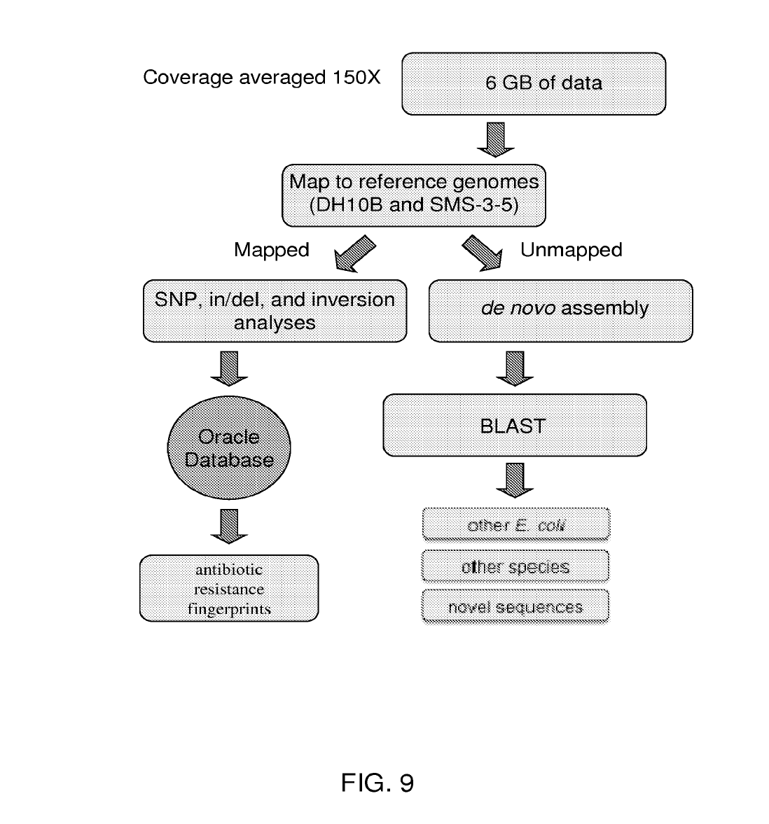

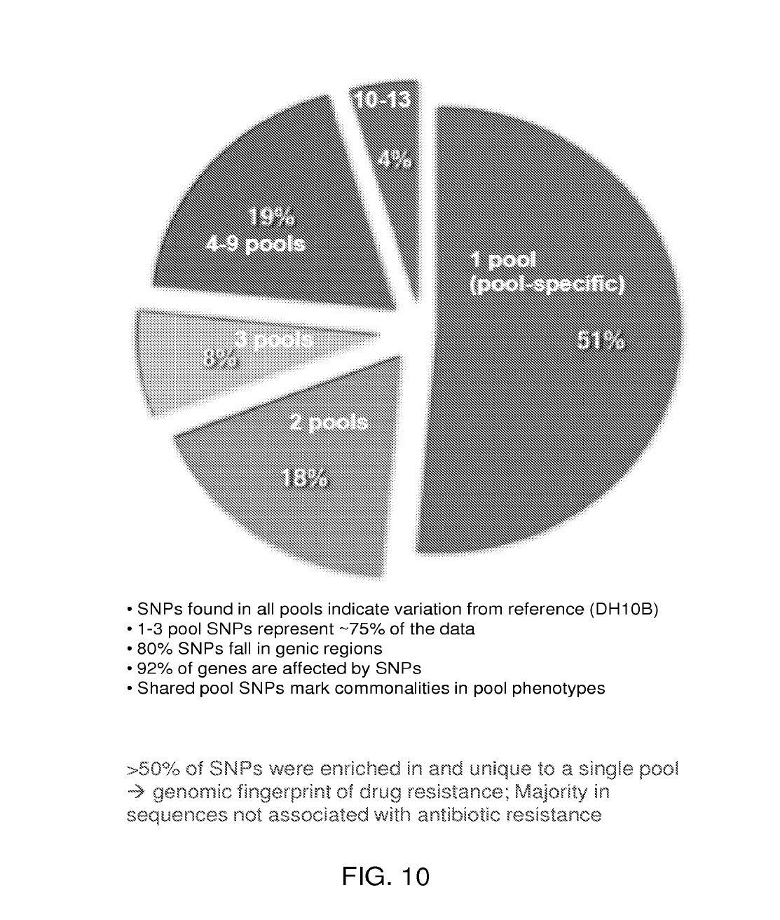

The present example demonstrates an exemplary workflow for assaying a cluster os strains for a particular phenotype, such as antibiotic resistance. FIG. 7 shows an antibiogram (outcome of testing for the sensitivity of an isolated bacterial strain to different antibiotics) related to fluoroquinolone (minimum inhibitory concentrations of certain amounts) and encompassing exemplary antibiotics (ciprofloxacin, gatifloxacin, levofloxacin, and norfloxacin). In FIG. 8, singletons were removed and those with missing data were removed to leave 16 pools. Sequencing of genomic data may occur by SOLiD sequencing. In FIG. 9, there is an exemplary sequence analysis strategy. In FIG. 10, illustrated are SNPs relative to the exemplary drug-susceptible strain DH10B. FIG. 11 shows exemplary SNPs associated with antibiotic resistance. High quality (HQ) SNPs, in some embodiments, is defined as those that occur 100% across the pool and the position and are the same base change. In FIG. 11, each pool was mapped to DH10B. The inventors subtracted SNPs that occurred in the sensitive pools (which were remarkably similar to each other). FIG. 11 shows the frequency of SNPs occurring in a 2-3 kB region of the genome, wherein peaks indicate regions that are high variable.

Therefore, in this embodiment illustrating an exemplary approach, a pooling strategy leverages information about antibiotic resistance phenotypes, and there is extremely high coverage, quality, and accuracy afforded, for example by SOLiD technology. For exemplary SNP analysis, one can identify and validate SNPs associated with each pool. In this particular case, most genic SNPs and all non-genic SNPs were not previously associated with antibiotic resistance. 92% of the genes were affected by SNPs, having an enrichment of carbohydrate metabolism genes. In this specific embodiments, clinical isolates with “record” high fluoroquinolone MICs match the exemplary soil isolate, SMS-3-5. Embodiments of the invention provide genomic fingerprints for drug resistance, but also drug susceptibility.

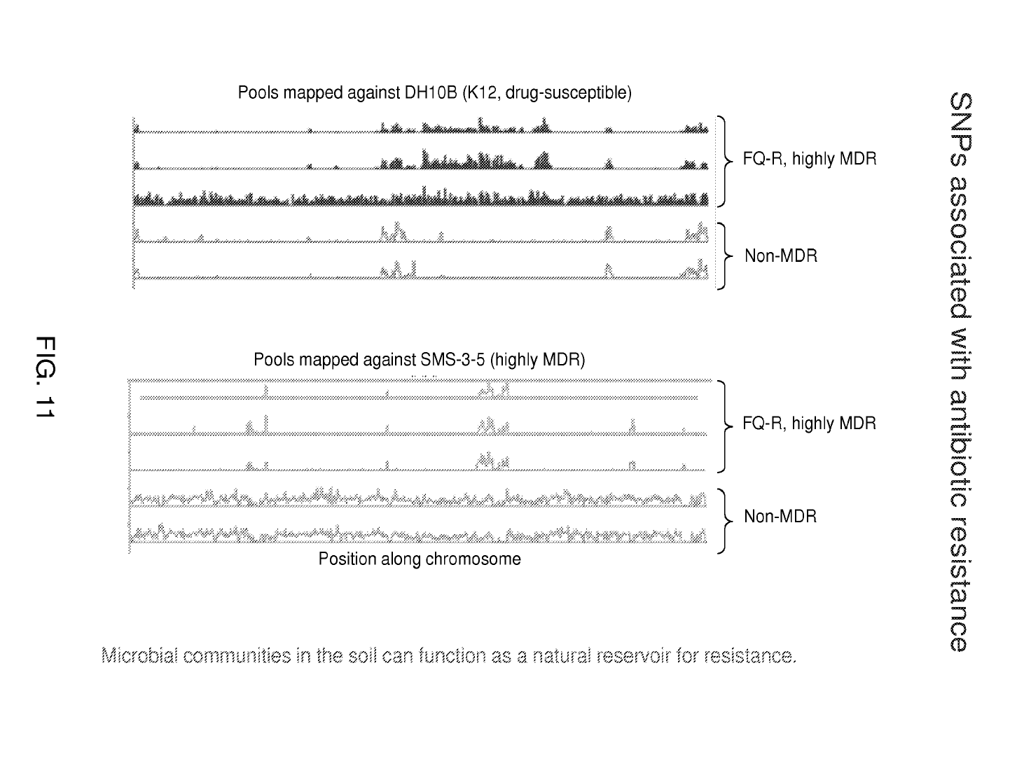

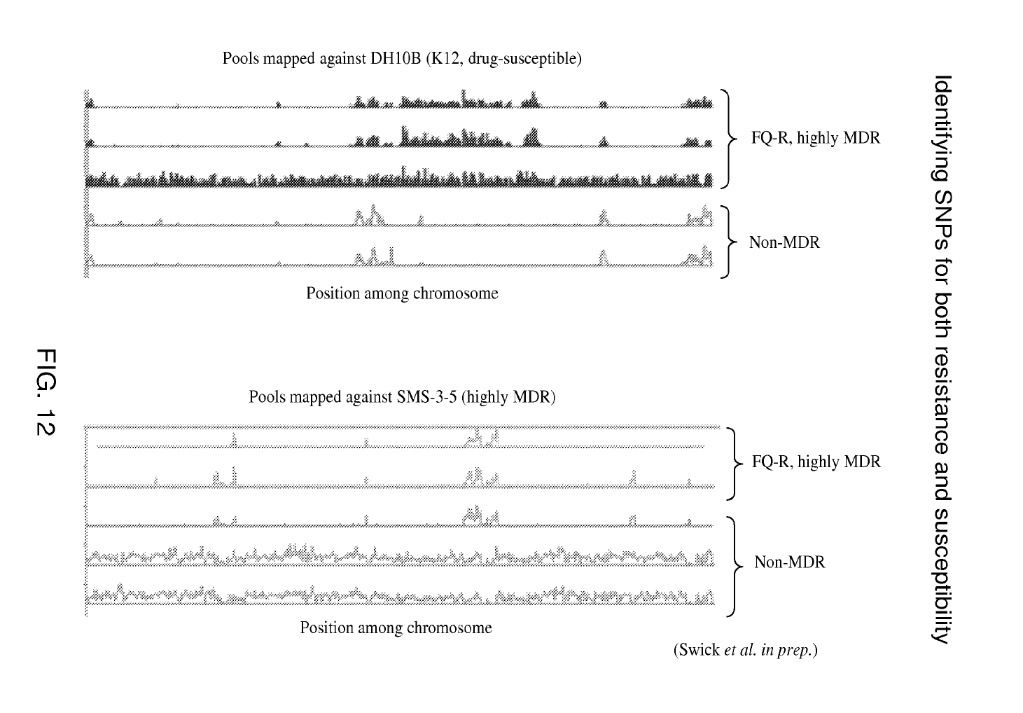

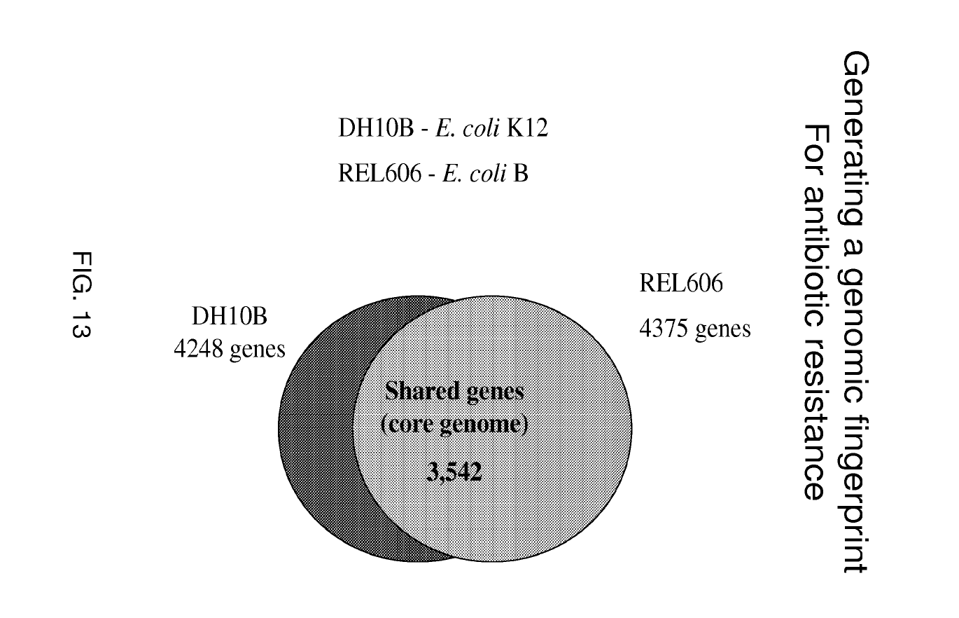

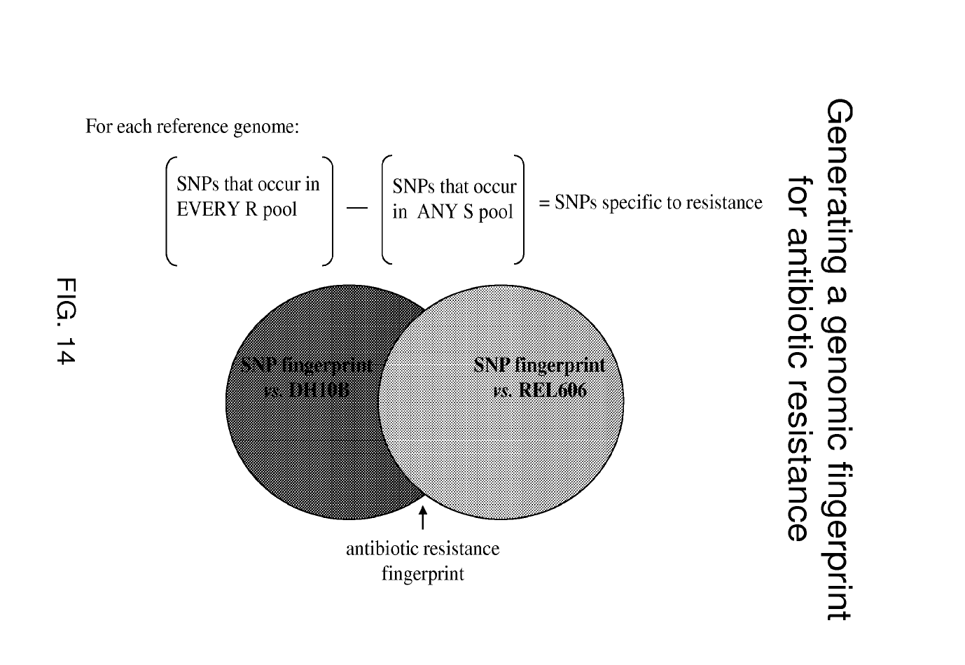

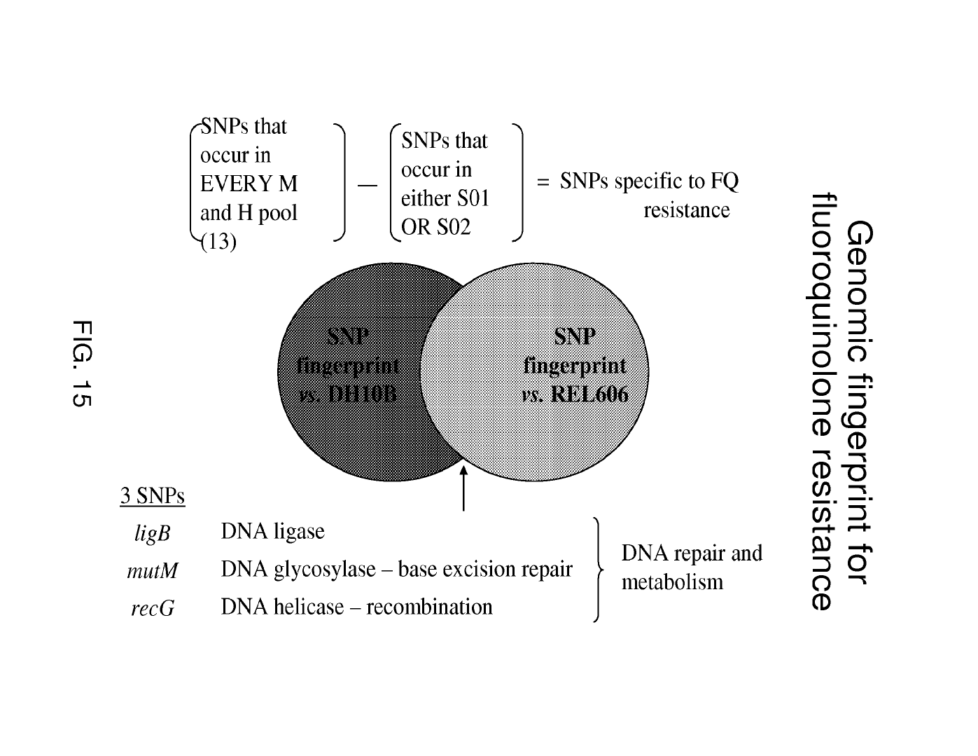

As an illustration of an embodiment of the invention, FIG. 12 shows identifying SNPs for both resistance and susceptibility to an exemplary antibiotic, FQ. FIG. 13 shows an example of two E. coli strains sharing a core set of genes, and FIG. 14 shows an example of generating a genomic fingerprint for antibiotic resistance. FIG. 15 provides an exemplary genomic fingerprint for fluoroquinolone resistance. If one takes SNPs that occurred unambiguously in every one of the 13 FQ-R pools, and subtract from that set any SNP that occurred in either of the FQ-S pools, one can get a short list of SNPs specific to FQ resistance. By generating this genomic fingerprint against each of the reference genomes, one can determine which SNPs were in common to both. For this set in FIG. 15, there are only 3 non-synonymous SNPs that passed this exemplary set of standards.

### Example 7

### Single Nucleotide Polymorphisms in the ligB, mutM, and recG Genes Accompany the Well-Known gyrAS83L Allele Associated with Fluoroquinolone Resistance

Drug-resistant bacterial infections are a worldwide problem that cause hundreds of thousands of deaths and cost billions of dollars each year. Many fluoroquinolone resistance mechanisms have been uncovered and the single nucleotide changes in the gyrA gene have been known for >30 years. However, known fluoroquinolone resistance mechanisms fail to explain why some bacteria resist concentrations of fluoroquinolones six orders of magnitude higher than normal and are also highly resistant to other antibiotic classes, suggesting that there may be additional genotypic changes. The inventors combined pooling, next generation sequencing, and SNP subtraction approaches to identify SNPs associated with fluoroquinolone resistance in bacterial pathogens. Using k-means clustering, 164 Escherichia coli clinical isolates collected over a decade and representing a broad spectrum of antibiotic resistance phenotypes were grouped into 16 pools based on similarity in susceptibility to 24 antibiotics. High quality (average coverage of 150×; P=10⁻¹⁹) SOliD sequencing data were generated for each pool and mapped to E. coli reference genomes. On the whole genome level, consensus sequences of drug-susceptible pools were highly similar to the drug-susceptible laboratory strain, DH10B, whereas those of multidrug-resistant pools were highly similar to the multidrug-resistant environmental strain, SMS-3-5. The remaining pools shared similarities to both DH1 OB and SMS-3-5. The inventors created a new computational platform that performs arbitrary set arithmetic to subtract SNPs occurring in any fluoroquinolone-susceptible isolates from those occurring in all fluoroquinolone-resistant isolates and vice versa. Relative to SMS-3-5, the inventors identified SNPs in common among all fluoroquinolone-susceptible isolates. Relative to both DH1 OB and REL606 (another drug-susceptible strain but from a different lineage), SNPs in common among all fluoroquinoloneresistant isolates fell within the genes gyrA, figB, mutM, and recG. Bioinformatic analysis revealed that the SNPs in the genes figB, mutM, and recG are tightly linked not only across the E. coli pan-genome but also throughout the order Enterobacteriales. Just like in all 144 of the fluoroquinolone-resistant clinical isolates, 11/12 strains in GenBank® that have the gyrAS83L SNP also have the other three SNPs, a remarkable 92% linkage. The one strain with gyrAS83L without all three had two of the SNPs. These data indicate that, in specific embodiments, variants in figB, mutM, and recG promote the emergence of fluoroquinolone resistance and also provide a rapidly assessed genomic fingerprint for diagnosis of fluoroquinolone-resistant infections.

### Example 8

### Conserved Genomic Variations in E. coli Clinical Isolates Correlate with Fluoroquinolone Resistance Phenotypes

In the present Example, the inventors demonstrate a combined pooling, sequencing, and SNP-subtraction based approach to identify SNPs associated with fluoroquinolone resistance and susceptibility in bacterial pathogens. The inventors grouped 164 Escherichia coli clinical isolates into 16 pools based on similarity in patterns of susceptibility or resistance to 21 antibiotics by k-means clustering. SOliD sequencing data were generated for each pool and compared to selected E. coli reference genomes. Variations at the whole genome level of the drug-susceptible pools aligned to the genome of the exemplary drug-susceptible laboratory strain, DH1 DB, whereas those of multidrug-resistant pools were more similar to the exemplary multidrug-resistant environmental strain, SMS-3-5. DH1 DB and SMS-3-5 represent the two extremes seen in the collection and the rest of the pool sequences fell between these two extremes. The inventors have isolated putative genomic fingerprints of fluoroquinolone susceptibility as well as fluoroquinolone resistance. Relative to both DH1 DB and another drug-susceptible strain of a different lineage, REL606, SNPs encoding nonsynonymous changes in protein sequences in common among all pools of fluoroquinolone-resistant isolates fell within the gyrA, ligB, mutM and recG genes—all of which are involved in DNA metabolism. These alleles of ligB, mutM, and recG are tightly linked in the E. coli pan-genome and the gyrA variant occurs only when accompanied by two or three of these variants, indicating that they may be involved in the evolution of fluoroquinolone resistance.

### Example 9

### Clinical Isolate Selection and Pooling Strategy

The inventors have taken advantage of a curated collection of >4,000 E. coli clinical isolates^{7,8} and associated susceptibility data for antibiotics from all the major drug classes (see Table 1). They selected 164 non-clonal isolates, each from a patient occurring uniquely in the set, which represented all of the antibiotic resistance phenotypes existing in the entire collection. These isolates ranged from susceptible to all tested antibiotics to multidrug resistant, and had measured fluoroquinolone minimal inhibitory concentrations (MIC) spanning six orders of magnitude⁷. They used the MIC values for four fluoroquinolones⁷ and the susceptibility status to 17 additional antibiotics as parameters in k-means clustering using Cluster 3.0⁹ to group the isolates into 16 pools. The number of isolates in each pool ranged from 2-33 strains. Table 3 describes the pools and their consensus resistance phenotypes. Because of the multiple parameters used in the clustering algorithm, only a rough ordering of the pools can be easily described. Two pools made up of nine isolates each were fluoroquinolone susceptible (liS″) and also susceptible to most other antibiotics. Three pools were made up of multidrug-resistant (resistant to antibiotics in ≧3 separate drug classes¹⁰) isolates with high fluoroquinolone MICs (“H”). The remaining pools (“M”) were intermediate between the other two sets, and were designated numerically (randomly) in the context of antibiotic resistance.

### Example 10

### Sequencing, SNP Identification, and Genome Comparison

Besides decreasing cost, sequencing pools of isolates results in internal normalization, dampening non-specific sequence variation from individual strains while highlighting conserved genetic variants. The inventors subjected pools of clinical isolate genomic DNA to next generation sequencing (NGS) on the ABI SOliD 3 Platform (Life Technologies) and mapped the resulting data to three E. coli reference genomes: the well-annotated laboratory strain DH1 OB derived from the K-12 lineage¹¹, the ancestral REL606 strain from the B lineage¹², and the highly multidrug-resistant environmental isolate SMS-35¹³. In addition to their antibiotic resistance status, other factors were considered in choosing these strains as references. DH1 OB is among the best-annotated, highly studied strains in GenBank®; REL606 is the subject of a long-term evolution experiment^{14,15}; and SMS-3-5¹³ is among the most diverged strains from DH1 OB, as measured by hierarchical analysis of the strains in GenBank®.

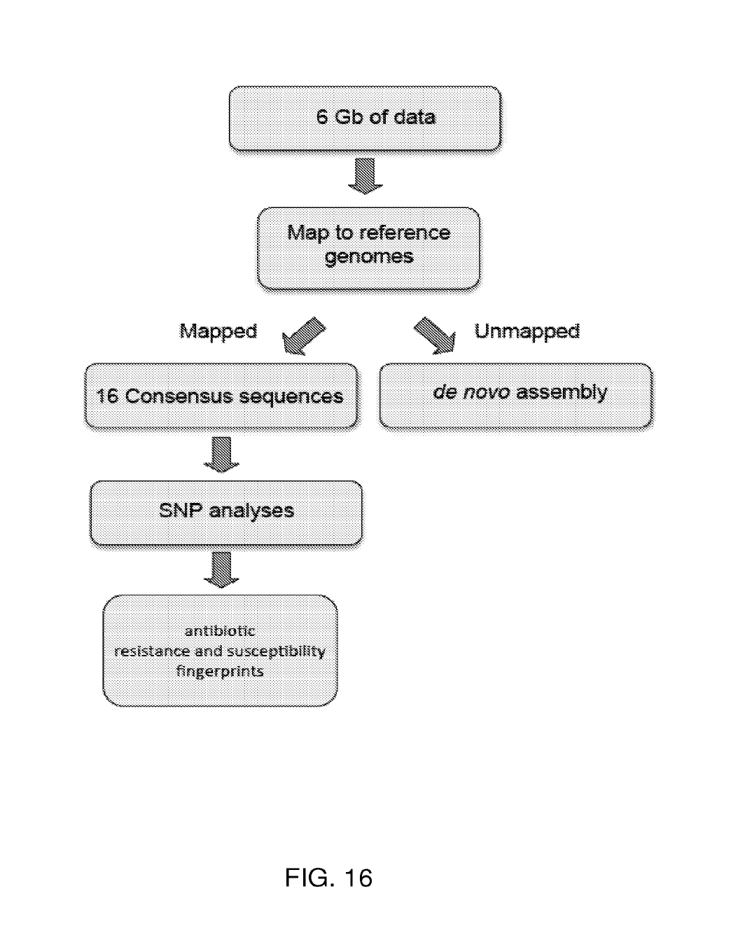

Reads were processed, mapped against each reference genome, and single nucleotide polymorphisms (SNPs) were called (FIG. 16 shows an exemplary data workflow). The coverage per base ranged from 20-400×, averaging 150×). More than half the reads mapping to anyone particular base identified as a difference against the reference genome was sufficient to call that position a SNP. A number of SNPs were called as identical in both nucleotide position and base identity for all reads from a pool, but differed from the reference; the inventors refer to these as unanimous SNPs. Mixed SNPs are those that did not pass this level of stringency.

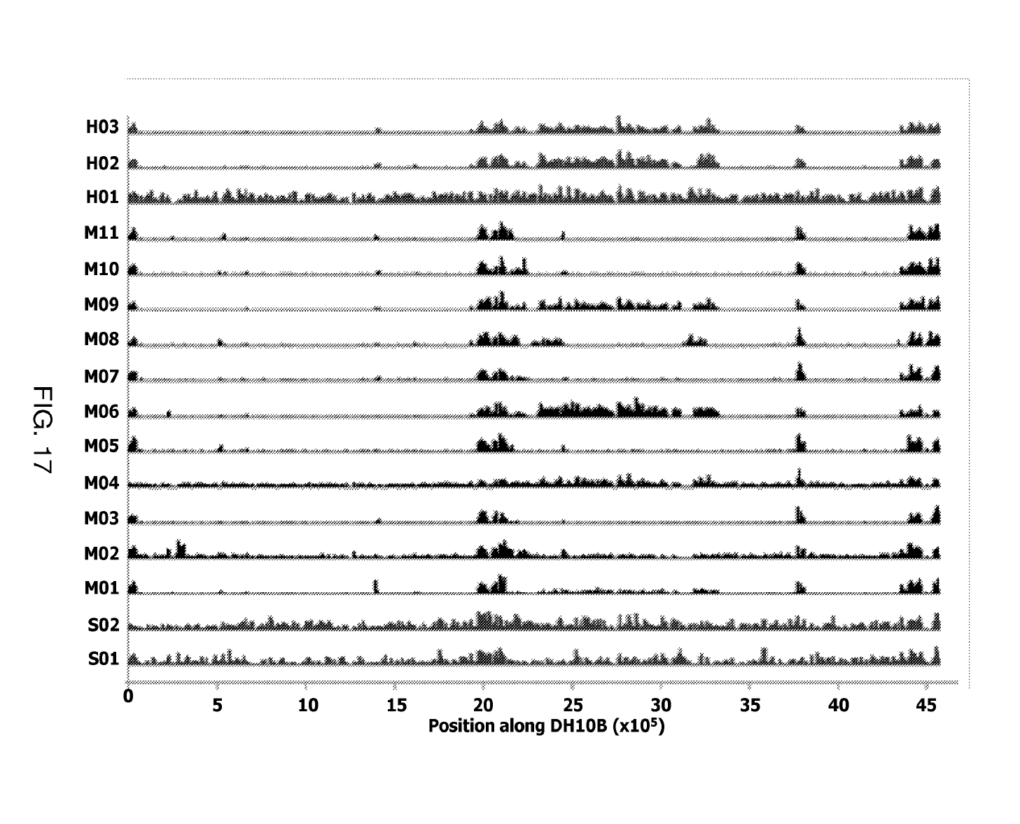

Mapped against the 4.6 Mb DH1 OB reference genome, reads from all 16 pools identified unanimous and mixed SNPs at 1,135,007 loci; 80% of these SNPs were in coding regions, located in 92% of the 4,357 annotated genes¹¹ (see FIG. 4a). Unanimous SNPs measured with high confidence (p<1.0×10⁻¹⁹) accounted for 64,773 loci. For the remainder of the analyses in this paper, the inventors focused on unanimous SNPs. The number of unanimous SNPs detected in each pool varied and were not uniformly distributed on the DH1 OB chromosome (see FIG. 17). Despite the differences in numbers of isolates in the pools, the quantity of unanimous SNP calls in each pool did not correlate with the pool size (see FIG. 4b). This finding demonstrates the usefulness of sequencing pools of large numbers of bacterial genomes with this platform. The sequence data from one pool of two isolates (H01) diverged extremely from all the reference genomes; the inventors excluded this pool from the further SNP analysis.

The inventors used qPCR allelic discrimination assays as independent corroboration of the NGS-based SNP discovery. They chose four candidate SNPs detected in three separate pools and tested their frequency among individual isolates of the pools. Allelic frequencies were consistent with predictions based on NGS mapping.

The inventors used the 5.1 Mb SMS-3-5 genome as the reference, mapping SNPs on 1,450,796 loci. More SNPs were identified in pools containing the fluoroquinolonesusceptible, non-MDR isolates (107,395 loci in S01, and 94,543 S02, respectively), and these were distributed throughout the chromosome. In contrast, pools containing isolates with high fluoroquinolone MICs and exhibiting multidrug resistance mapped SNPs to fewer loci (81,403 in H03; 88,677 in M11), consistent with the model that the genomes in these pools had more in common with the phenotypically similar SMS-3-5. These SNPs were clustered on loci that were also regions of high variation in multiple pools (highlighted regions in FIG. 6a). Comparing the DH1 OB and SMS-3-5 genomes lines up these loci with breakpoints of large-scale inversions and duplications in the evolution of the E. coli chromosome (see FIG. 6b); thus, these clusters may mark regions of genomic instability. The number of SNPs is a direct measure of the differences between a test sequence and the reference. The log ratio of the number of unanimous SNPs from each pool measured against either DH1 OB or SMS-3-5 (FIG. 5a) reveals the similarity of the pool to either of the reference sequences (FIG. 5b). The genomes of the clinical isolates of E. coli in the H02 and H03 pools (very high fluoroquinolone MICs and multidrug-resistant) were more similar to the environmental isolate than to the susceptible laboratory strain. The clinical strains may have inherited drug resistance mechanisms from environmental bacteria that serve as reservoirs for antibiotic resistance^{3,16}.

The inventors refined this analysis by comparing the number of unanimous SNPs in common between any two pools relative to every other pool. The commonality of SNPs is a similarity metric that defines a distance map between pools, and this can be represented as a hierarchical tree (FIG. 5c). The branch length of each tree in the axes of the plot denotes their unrooted relative distance in the hierarchical clustering process. The dot plot illustrates that the most susceptible pools and the most resistant pools clustered together, regardless of which reference genome was used. One pair of multidrug-resistant pools (M5 and M11) also clustered together. The dispersed nature of the remaining pools suggests that polymorphisms occur in different regions of DH1 OB and SMS-3-5, as also evidenced by the SNP distribution graphs (see FIG. 6a and FIG. 17). Thus, phenotypic differences between the two reference genomes are not a consequence of variations in a core set of genes, but are dispersed among multiple different loci.

### Example 11

### Genomic Fingerprints of Fluoroquinolone Resistance and Susceptibility

Among all discovered unanimous SNPs, homotypic changes (purine to purine or pyrimidine to pyrimidine) were detected twice as frequently as heterotypic SNP conversions, in agreement with most metazoan and human¹⁷ data although there is at least one counter example¹⁸. Unexpectedly, 99.2% of the SNPs discovered were biallelic—meaning that only two of the four bases were found at those positions. The few remaining SNPS were triallelic. At no single position did all four nucleotides occur. The high frequency of biallelism means that direct subtraction of SNPs between the pools of varying phenotypes will reveal SNPs directly linked to specific antibiotic resistance traits. The inventors report this analysis on resistance or susceptibility to fluoroquinolones because the strain dataset is largest and best characterized for that antibiotic class^{7,10,19}. With additional but routine analysis, one can apply this approach for studying resistance to additional antibiotics.

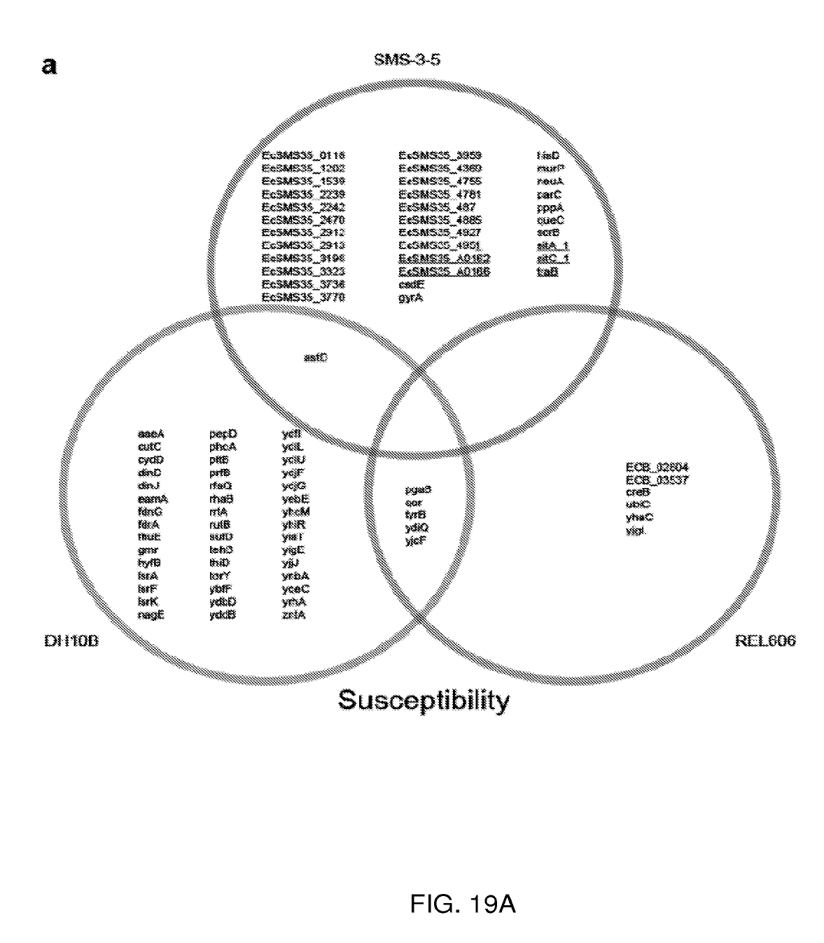

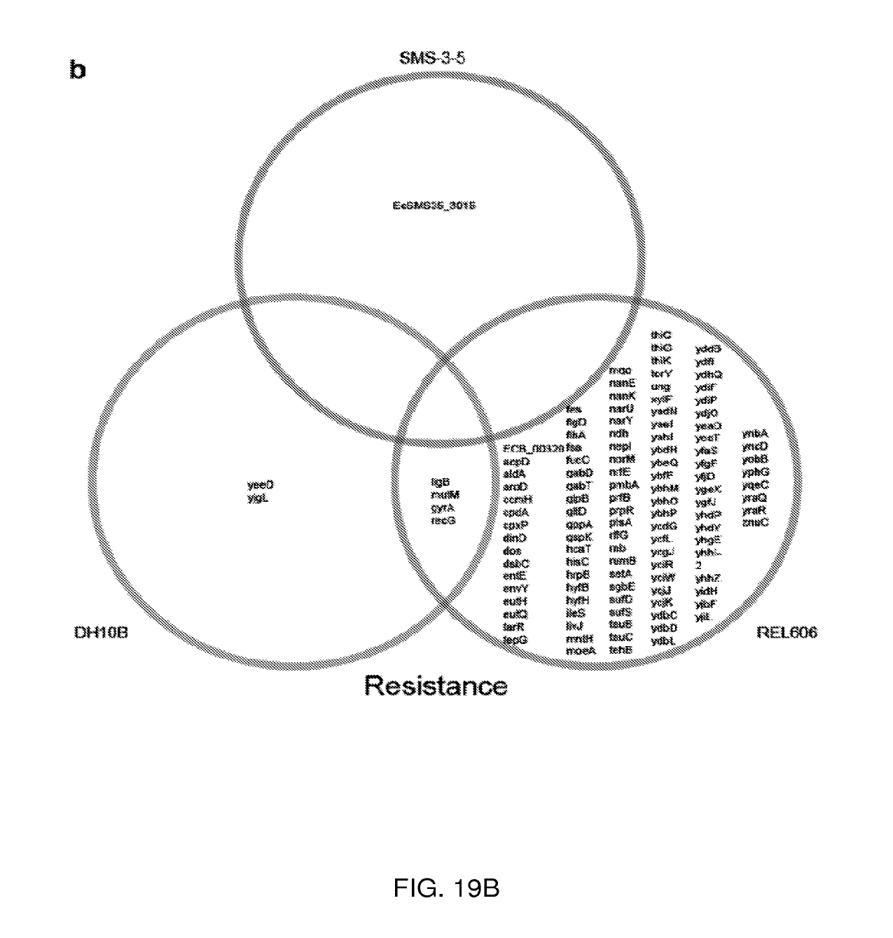

About half of all the unanimous SNPs were pool-specific (FIG. 4c), and the remaining SNPs were shared between at least two pools; 1.6% of the SNPs were found in all 13 fluoroquinolone-resistant pools. The inventors created a new computational platform that performs arbitrary set arithmetic to enrich for SNPs linked to fluoroquinolone susceptibility or resistance. To their knowledge, this analysis is the first time SNP subtraction has been carried out on pooled data. Unanimous SNPs were mapped to both coding and noncoding regions; however, the subset that results in nonsynonymous changes to annotated genes provides insight into mechanisms of antibiotic susceptibility and resistance. To identify genomic signatures linked to antibiotic susceptibility, they identified unanimous SNPs on loci in common between the two fluoroquinolone-susceptible pools (S01 and S02), but that did not occur in any of the moderate or highly fluoroquinolone-resistant pools; this process was repeated for all three, reference genomes. The inventors identified the annotated genes for which the encoded proteins were predicted to change in the presence of the SNP, and determined their commonality between the different reference genomes (FIG. 19).

The subtraction identified 35 genes in fluoroquinolone-susceptible pools that differed from SMS-3-5 (FIG. 19a). Five of these genes are encoded on plasmid pSMS35_{—}130, and the rest on the main chromosome. Of the thirty chromosomal genes, one is also variant against DH1 OB. Although a core set of annotated genes are shared among the three reference genomes used, exclusivity in the Venn diagram may represent variations linked to the specific phenotype of the reference used, or be a consequence of a gene being absent in the annotation of either of the two other genomes. Nonetheless, in agreement with our published data¹⁹, only “wild-type” alleles of gyrA (one of two genes encoding gyrase) and parC (one of two genes encoding topoisomerase IV) were found in the fluoroquinolone-susceptible pools. The same “wildtype” alleles, namely the S83 allele of gyrA and the S80 allele of parC are also ubiquitous in sequenced genomes of fluoroquinolone-susceptible E. coli isolates banked in GenBank® and the Broad Institute.

The inventors performed a similar subtraction to identify genomic variants linked to fluoroquinolone resistance. Relative to the reference genome of DH1 OB, 230 unanimous SNPs in both coding and noncoding regions were shared among all the fluoroquinolone-resistant pools, but were absent from the fluoroquinolone-susceptible pools. Six of these SNPs resulted in non-synonymous changes in annotated protein coding genes. Using REL606 as a reference genome, 989 SNPs conformed to the same fluoroquinolone-resistance based criteria; 117 of these result in non-synonymous gene variations. Using SMS-3-5 as a reference, the only genic SNP exhibiting unanimous, nonsynonymous variation was in EcSMS35_{—}3015, a locus encoding a xanthine/uracil permease family protein very similar in sequence to the ygfO gene.

The resulting subtractions are summarized in FIG. 19b, which illustrates the variant genes among the three reference genomes in a Venn diagram. The four genes exhibiting nonsynonymous changes relative to both DH1 OB and REL606, but are in agreement with SMS-3-5, represent putative SNP fingerprints of fluoroquinolone resistance (see FIG. 19a, genes contained within both of the bottom circles). One of these SNPs results in the S83L variant in gyrA, which is in all the fluoroquinoloneresistant isolates¹⁹, but the polymorphisms in ligB, encoding a DNA ligase; mutM, encoding a DNA glycosylase; or recG, encoding a DNA helicase, have not been associated with fluoroquinolone resistance. However, a deletion in recG was reported to cause increased fluoroquinolone-susceptibility²⁰.

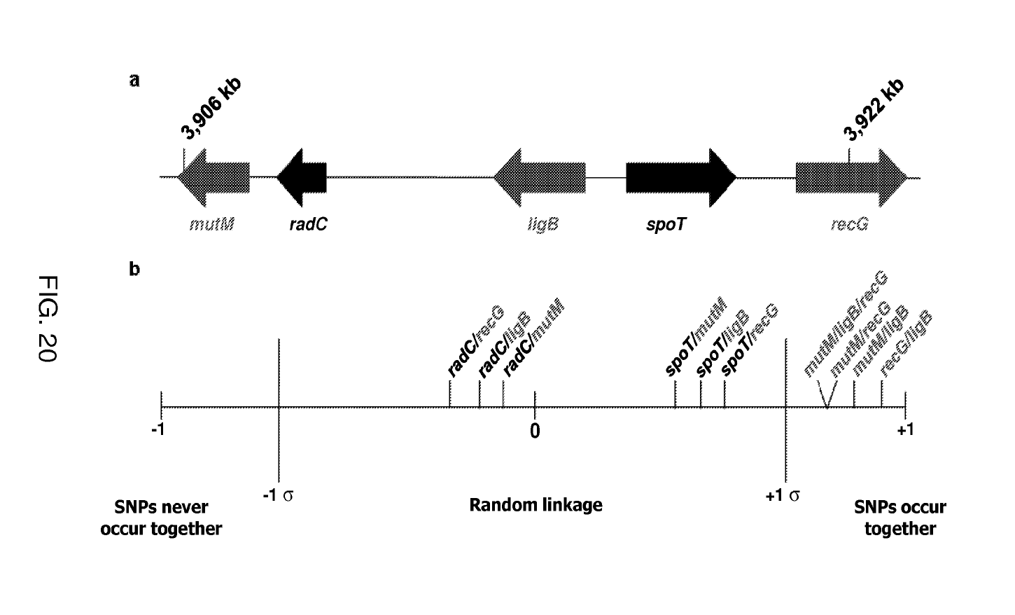

The ligB, mutM, and recG genes are encoded in a 15 kb cluster on the E. coli chromosome (FIG. 20a), some distance from gyrA. Linkage of variants among these three genes may be explained by their close chromosomal proximity through coinheritance. To investigate this linkage outside of the sequenced pools, the inventors analyzed the sequences of 39 fully annotated E. coli genomes archived in GenBank® and 83 annotated draft genomes from the Broad Institute (Escherichia coli Antibiotic Resistance Sequencing Project, Broad Institute of Harvard and MIT (see world wide website), which represent a wide range of environmental conditions. The polymorphisms in the three loci in this cluster were tightly linked, with 109 of the 122 strains encoding none or all three of the SNPs (FIG. 20b).

If polymorphisms in these genes were linked through co-inheritance, other SNPs found within the same genomic cluster should exhibit similar linkage. The inventors analyzed the linkage of these SNPs to variations found in the spoT locus, located between figB and recG; and variations in radC, located between mutM and figB. Linkage of either spoT or radC with mutM, recG, or figB did not differ from what would be expected by random chance (FIG. 20b). These results indicate that the tight linkage of the mutM, figB, and recG SNPs is under positive evolutionary selection independent of the physical linkage.

In addition to the gyrA S83L variant, variants of the gyrA gene (D87Y/N) and the parC gene (S801 and E84K/G) occur frequently in fluoroquinolone-resistant E. coli⁷. A unanimous SNP in pool M03 (resistant only to fluoroquinolones) maps to the parC S801 variant, but was mixed in composition in other pools containing antibiotic-resistant isolates. This result implies that in the absence of resistance mechanisms to other antibiotics, the additional parC S801 variant may be necessary for clinically relevant fluoroquinolone resistance. The other gyrA and parC variants mentioned above were found in the fluoroquinolone-resistant pools at high frequency, but were not found unanimously in any of the pools.

### Example 12

### Presence of Cryptic Prophage in Clinical Isolates of E. coli

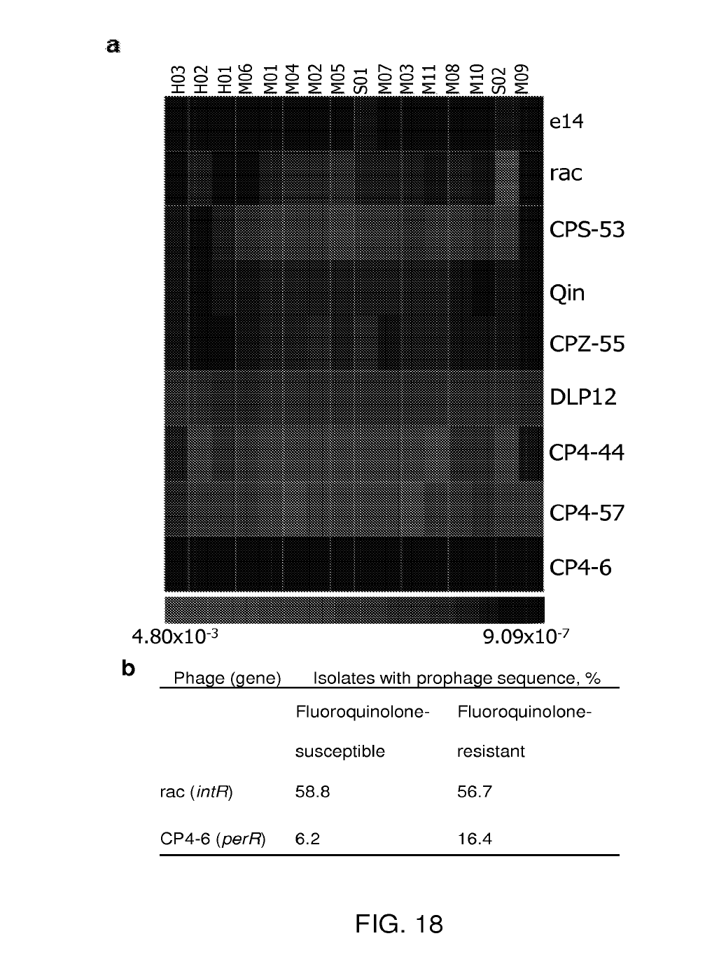

Wang et al. found that deletion of any of three cryptic prophages (rac, e14, and CP4-6) lowered nalidixic acid MICs in the E. coli BW25113 K-12 strain²¹. Despite very good mappability and the very high coverage of the pooled sequences, these prophage sequences were detected infrequently and only in some isolates in most pools (FIG. 18a). Whereas the prophage sequences CP4-44 and CP4-57 were detected in at least some isolates in a majority of pools, and there was no correlation with fluoroquinolone resistance. PCR screening for the presence of intR for rac and perR for CP-4-6 verified that these sequences were found equally in fluoroquinolone-susceptible and fluoroquinolone-resistant clinical isolates (see FIG. 18b). Thus, in spite of affecting quinolone MICs, these prophage sequences are not correlated with fluoroquinolone resistance in clinical isolates.

### Example 13

### Significance of Certain Embodiments of the Invention

The phenotype-based pooling and subtraction approach to whole genome sequencing methods the inventors developed to examine antibiotic resistance in E. coli can be used to probe SNPs associated with any phenotype for which large enough sequence datasets exist. Similar to many human diseases (e.g. diabetes, cancer), antibiotic resistance is polygenic and involves multiple genetic loci. The antibiotic resistance phenotype for any one E. coli isolate in the different human microbiomes is the sum effect of the different genomic variations that contribute directly or indirectly to antibiotic susceptibility or resistance. The clinical isolates the inventors sampled are a non-random mosaic of genotypes with corresponding phenotypes between the two extremes DH1 OB/REL606 and SMS-35.

The two MDR strains that make up pool H01 were physiologically identified as E. coli in standard tests but the genome sequences were clearly divergent from all three of the reference E. coli genomes used in embodiments of the invention. As such, they push the species boundary for E. coli. As more bacterial genomes are sequenced, the current concept of species will likely be additionally challenged.

Origin of Antibiotic Resistance

Although the ligB, mutM, and recG SNPs are shared by all fluoroquinolone-resistant clinical isolates in embodiments of the invention, this trio is also found in many strains not known to be resistant to fluoroquinolones. Thus, these variants in DNA metabolism may serve as a potentiating genetic background to evolve mechanisms of fluoroquinolone resistance without being directly involved; they may be required for the gyrA S83L variant to occur. Indeed, in the GenBank® and Broad Institute curated E. coli genome sequences, only one case (out of 10) of the S83L gyrA variant was not linked to the SNP trio, but even this one was still linked to two (figB and recG) of the three SNPs.

Although antibiotic resistance is generally thought to be the consequence of genetic alteration, the finding that some fluoroquinolone-resistant clinical isolates are more highly related to an MDR environmental isolate led the inventors to search for SNPs associated with fluoroquinolone susceptibility. The concept of genetic variations associated with antibiotic susceptibility is distinct from a conventional view in which susceptibility is a default state upon which drug resistance variations are layered. Detecting the set of genomic variants linked to antibiotic susceptible or resistant phenotypes serves as the basis of a rapid diagnostic to guide clinicians to the selection of an appropriate antibiotic regimen. Such a diagnostic would minimize empirical antibiotic prescription, maximize treatment efficacy, and extend the useful life of the existing antibiotic arsenal.

### REFERENCES

All publications mentioned in the specification are indicative of the level of those skilled in the art to which the invention pertains. All publications are herein incorporated by reference to the same extent as if each individual publication was specifically and individually indicated to be incorporated by reference.

1. Boucher, H. W. et al. Bad bugs, no drugs: no ESKAPE! An update from the Infectious Diseases Society of America. Glin. Infect. Dis. 48, 1-12 (2009).

2. Mauldin, P. O., Salgado, C. D., Hansen, I. S., Durup, D. T. & Bosso, J. A. Attributable hospital cost and length of stay associated with health care-associated infections caused by antibiotic-resistant gram-negative bacteria. Antimicrob. Agents Ghemother. 54, 109-115 (2010).

3. Davies, J. & Davies, D. Origins and evolution of antibiotic resistance. Microbial. Mol. Biol. Rev. 74, 417-433 (2010).

4. Taubes, G. The bacteria fight back. Science 321, 356-361 (2008).

5. Pitout, J. D. D. & Laupland, K. B. Extended-spectrum beta-lactamase-producing Enterobacteriaceae: an emerging public-health concern. Lancet Infect Dis 8, 159166 (2008).

6. Nikaido, H. Multidrug resistance in bacteria. Annu. Rev. Biochem. 78, 119-146 (2009).

7. Becnel Boyd, L. et al. Relationships among ciprofloxacin, gatifloxacin, levofloxacin, and norfloxacin MICs for fluoroquinolone-resistant Escherichia coli clinical isolates. Antimicrob. Agents Ghemother. 53,229-234 (2009).

8. Boyd, L. B. et al. Increased fluoroquinolone resistance with time in Escherichia coli from >17,000 patients at a large county hospital as a function of culture site, age, sex, and location. BMG Infect. Dis. 8, 4 (2008).

9. Eisen, M. B., Spellman, P. T., Brown, P. O. & Botstein, D. Cluster analysis and display of genome-wide expression patterns. Proc. Nat!. Acad. Sci. U.S.A. 95, 14863-14868 (1998).

10. Swick, M. C., Morgan-Linnell, S. K., Carlson, K. M. & Zechiedrich, L. Expression of multidrug efflux pump genes acrAB-toIC, mdfA, and norE in Escherichia coli clinical isolates as a function of fluoroquinolone and multidrug resistance. Antimicrob. Agents Ghemother. 55, 921-924 (2011).

11. Durfee, T. et al. The complete genome sequence of Escherichia coli DH1 OB: insights into the biology of a laboratory workhorse. J. Bacterial. 190, 2597-2606 (2008).

12. Jeong, H. et al. Genome sequences of Escherichia coli B strains REL606 and BL21 (DE3). J. Mol. Biol. 394, 644-652 (2009).

13. Fricke, W. F. et al. Insights into the environmental resistance gene pool from the genome sequence of the multidrug-resistant environmental isolate Escherichia coli SMS-3-5. J. Bacterial. 190, 6779-6794 (2008).

14. Barrick, J. E. et al. Genome evolution and adaptation in a long-term experiment with Escherichia coli. Nature 461, 1243-1247 (2009).

15. Daegelen, P., Studier, F. W., Lenski, R. E., Cure, S. & Kim, J. F. Tracing ancestors and relatives of Escherichia coli B, and the derivation of B strains REL606 and BL21 (DE3). J. Mol. Biol. 394, 634-643 (2009).

16. D'Costa, V. M., McGrann, K. M., Hughes, D. W. & Wright, G. D. Sampling the antibiotic resistome. Science 311, 374-377 (2006).

17. Zhang, Z. & Gerstein, M. Patterns of nucleotide substitution, insertion and deletion in the human genome inferred from pseudogenes. Nucleic Acids Res. 31, 5338-5348 (2003).

18. Keller, I., Bensasson, D. & Nichols, R. A. Transition-Transversion Bias Is Not Universal: A Counter Example from Grasshopper Pseudogenes. PLoS Genet 3, e22 (2007).

19. Morgan-Linnell, S. K., Becnel Boyd, L., Steffen, D. & Zechiedrich, L. Mechanisms accounting for fluoroquinolone resistance in Escherichia coli clinical isolates. Antimicrob. Agents Chemother. 53,235-241 (2009).

20. Sutherland, J. H. & Tse-Dinh, Y.-C. Analysis of RuvABC and RecG involvement in the Escherichia coli response to the covalent topoisomerase-DNA complex. J. Bacteriol. 192, 4445-4451 (2010).

21. Wang, X. et al. Cryptic prophages help bacteria cope with adverse environments. Nat Commun 1, 147 (2010).

22. Blattner, F. R. et al. The complete genome sequence of Escherichia coli K-12. Science 277, 1453-1462 (1997).

23. NCClS Performance standards for antimicrobial susceptibility testing: ninth informational supplement. National Committee for Clinical Laboratory Standards (2002).

24. Huang, D. W., Sherman, B. T. & Lempicki, R. A. Systematic and integrative analysis of large gene lists using DAVID bioinformatics resources. Nat Protoc 4,44-57 (2009).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of determining resistance or susceptibility of one or more bacteria to one or more antibiotics, comprising the steps of:
obtaining or providing a plurality of bacteria of the same species;
sequencing a nucleic acid region from the plurality of bacteria;
comparing the sequence to the corresponding sequence of a reference bacteria of the same species, said reference bacteria known to be resistant or susceptible, respectively, to the one or more antibiotics; and
identifying differences, similarities, or both between the bacteria from the plurality with the reference bacteria.

2. A method of determining resistance or susceptibility of one or more bacteria to one or more antibiotics, comprising the steps of:
grouping a plurality of bacteria based on known patterns of susceptibility or resistance to one or more antibiotics;
sequencing nucleic acid from each of the bacteria in the plurality;
comparing the sequence of the nucleic acid to a corresponding nucleic acid sequence from a reference bacteria of the same species, said reference bacteria known to be resistant or susceptible, respectively, to the one or more antibiotics; identifying a genomic fingerprint for the plurality that represents a respective genotype for the susceptibility or resistance.

3. The method of claim 2, wherein the genomic fingerprint comprises one or more SNPs that are common among the plurality.

4. The method of claim 3, wherein the SNPs are located in DNA metabolism genes.

5. The method of claim 2, wherein the antibiotic is selected from the group consisting of aminoglycosides, ansamycins, carbacephem, carbapenems, cephalosporins, glycopeptides, lincosamides, lipopeptide, macrolides, monobactams, nitrofurans, penicillins, polypeptides, quinolones, fluoroquinolones, sulfonamides, tetracyclines, sulfa drugs, drugs against mycobacteria, and a combination thereof.

6. The method of claim 1, wherein information from the determination of resistance or susceptibility of a bacteria is employed in diagnosis of a pathogenic bacteria from an individual.

7. The method of claim 6, wherein the method further comprises obtaining a sample from the individual.

8. The method of claim 7, wherein the sample is mucus, sputum, saliva, feces, blood, nasal swab, throat swab, or a mixture thereof.

9. The method of claim 2, wherein the sequencing is further defined as next generation sequencing.
